(19) United States
(12) Patent Application Publication
(10) Pub. No.: US 2009/0112309 A1
(43) Pub. Date: Apr. 30, 2009

(54) Collapsible Heart Valve with Polymer Leaflets

(75) Inventors: Fernando Jaramillo, Miami, FL (US); Richard T. Schoephoerster, Miami, FL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: THE FLORIDA INTERNATIONAL UNIVERSITY BOARD OF TRUSTEES, Miami, FL (US)

(21) Appl. No.: 11/993,948

(22) PCT Filed: Jul. 21, 2006
(86) PCT No.: PCT/US2006/028296

§ 371 Date: Nov. 4, 2008

Related U.S. Application Data
(60) Provisional application No. 60/701,302, filed on Jul. 21, 2005.

(51) Int. Cl. A61F 2/06; A61F 2/24; A61B 19/00

(52) U.S. Cl. 623/1.26; 623/2.1; 623/1.15; 128/898

(57) ABSTRACT

A Catheter Based Heart Valve (CBHV) is described herein which replaces a non functional, natural heart valve. The CBHV significantly reduces the invasiveness of the implantation procedure by being inserted with a catheter as opposed to open heart surgery. Additionally, the CBHV is coated with a biocompatible material to reduce the thrombogenic effects and to increase durability of the CBHV. The CBHV includes a stent and two or more polymer leaflets sewn to the stent. The stent is a wire assembly coated with Polystyrene-Polyisobutylene-Polystyrene (SIBS). The leaflets are made from a polyester weave as a core material and are coated with SIBS before being sewn to the stent. Other biocompatible materials may be used, such as stainless steel, Titanium, Nickel-Titanium alloys, etc.

### RELATED APPLICATIONS

This patent application claims priority benefit of U.S. Provisional Patent Application No. 60/701,302, filed on Jul. 21, 2005, the entirety of which is hereby incorporated by reference.

### BACKGROUND

1. Field of the Disclosure

The present disclosure is generally directed to artificial heart valves, and more particularly to collapsible artificial heart valves that are deployed via a catheter.

2. Description of Related Art

The heart is the organ responsible for keeping blood circulating through the body. This task would not be possible if it was not for the action of valves. Four heart valves are key components that facilitate blood circulation in a single direction, and that the contraction force exerted by the heart is effectively transformed into blood flow.

Each time the heart contracts or relaxes, two of the four valves close and the other two open. There are two states of the heart: relaxed or contracted. Depending on the state of the heart, a heart valve has two specific functions: either to open smoothly without interfering blood flow or to close sharply to impede the flow in the opposite direction.

The anatomy of the heart allows it to simultaneously maintain the flow of the two major blood circuits in the body: pulmonary circulation and systemic circulation, which also includes the coronary circulation. This simultaneous action of keeping blood flowing through both circuits requires that the heart valves work in pairs, namely, the tricuspid and the pulmonary valve work together to direct the flow toward the lungs, and the mitral and aortic valves direct the flow toward the rest of the body including the heart.

From the two circulations, the systemic circulation is the one that demands most of the energy of the heart because it operates under higher pressures and greater flow resistance. Consequently, the left heart is more susceptible to valve disorders. This condition makes the aortic and mitral valves primary subjects of research.

According to the American Heart Association it is estimated that around 19,700 people in the United States die every year from heart valve disease, and another 42,000 die from different causes aggravated by valvular problems. During 1996, 79,000 heart valve replacements were carried out in the United States, a quantity that was reported to increase by 5,000 more replacements by 1997. Although improvement has been evident in this area of medical treatments, still a mortality rate between 30% and 55% exists in patients with valvular prostheses during the first 10 years after surgery.

The aortic valve, representing almost 60% of the valve replacement cases, is located at the beginning of the systemic circulation and right next to the coronary ostia. Once the aortic valve closes the oxygenated blood flows into the heart through the right and left coronary arteries.

The mitral valve, located between the left atrium and the left ventricle offers a different set of conditions. Although the mitral valve is not surrounded by any arterial entrances, it is located in a zone with greater access difficulties, and its anatomical structure contains a set of “leaflet tensors” called chordae tendinae.

The human application of prosthetic heart valves goes back to 1960 when, for the first time, a human aortic valve was replaced. Since then, the use of valvular implants has been enhanced with new materials and new designs.

The first mechanical valves used a caged-ball mechanism to control blood flow. Pressure gradients across the occluder-ball produced its movement to close or open the flow area. Even though this design performed the function of a valve, there were several problems associated with it: The ball geometry and the closing impact of the ball against the cage ring were both causes of large downstream turbulence and hemolysis. In addition to blood damage, obstruction to myocardial contraction and thrombogenic materials were also problems.

Several designs having new materials including disks or leaflets instead of balls, improved the hemodynamic performance and durability of the implants, but two critical aspects remain pending for better solutions: 1) the highly invasive surgery required to implant the prosthesis, and 2) the thrombogenic effect of the implant's materials.

Typically, mechanical heart valve prostheses are made from pyrolytic carbon or other prosthetic materials that require rigorous anticoagulant therapy because the risk of coagulation is higher over the surface of the prosthesis. The thrombogenic aspect has drawn the attention of many biomedical institutions towards the creation and study of more biocompatible materials.

The Cardiovascular Engineering Center (CVEC) at the Florida International University is one of these institutions. It is presently testing a triblock polymer (Polystyrene-Polyisobutylene-Polystyrene) known as SIBS, a synthetic material that shows high levels of biocompatibility. Such a synthetic material and method of coating a porous prosthesis are described in U.S. Patent Publication No. 2005/0055075, U.S. Pat. No. 5,741,331 and U.S. Pat. No. 6,102,933 to Pinchuck et al., each of which is hereby incorporated herein by reference.

U.S. Patent Publication No. 2005/0055075 describes a process of applying a biocompatible solution to a porous prosthesis including the steps of applying a solution of a biocompatible block copolymer, including one or more elastomeric blocks and one or more thermoplastic blocks. U.S. Patent Publication No. 2005/0055075 further describes using a series of solvents to precipitate the copolymer onto the support structure of the porous prosthesis. SIBS is the preferred class of elastomeric material for forming vascular prostheses.

Currently, prosthetic heart valve technology includes several designs with disks or leaflets integrated into a rigid stent. This rigid stent is generally surrounded by a sewing cuff which allows the surgeon to suture the interface between the cuff and the tissue. This procedure, however, is highly invasive and its materials generally have a negative thrombogenic effect.

Prosthetic heart valves with rigid stents require open heart surgery for implantation. During the implantation procedure the patient is maintained alive by a heart-lung machine while the surgeon sutures the device into the heart. Due to the highly invasive nature of this procedure, not all individuals suffering from heart valve disease are considered proper candidates.

In those cases where a heart valve replacement has been performed, the risk of coagulation of blood becomes higher over the surface of the prosthesis. Mechanical heart valve prostheses made from pyrolytic carbon or other prosthetic metals require rigorous anticoagulant therapy. In the case of prosthetic valves using animal tissues, the thrombogenic effect is not as severe as for mechanical valves, but durability is noticeably lower.

Catheter based heart valves (CBHV) are expected to address the mentioned problems through the use of a catheter delivery system. Catheter delivery allows the interventional radiologist to make a heart valve replacement without highly invasive surgery.

Current catheter technology has been proven to be successful in the treatment of some cardiovascular pathologies with the advantage of requiring less traumatic procedures. Some relatively simple conditions like aneurysms and stenosis are currently being treated using catheter based devices, but more complex conditions, like heart valve disease, remain a challenge.

The replacement of a diseased heart valve with a prosthetic device that does not require open heart surgery is a problem that pushes current catheter and stent technology to achieve higher standards of performance.

The most elementary attempts to create heart valves that could be implanted using catheters started by focusing on the aortic position and by fusing the existing models of endovascular stents with jugular segments of bovine tissue. The stent provided all the structural support, while the jugular segments were used to work as the actual valve. Among other reasons, the use of a jugular segment was preferred because of its convenient natural geometry: these segments already contain an embedded valve that could be easily attached to a stent by sewing, but as expected, this concept was too simple to satisfy the anatomical details of the aortic position. Once the valve was implanted, either the coronary orifices were blocked, or the device migrated through the aorta.

Another concept developed to improve some of the deficiencies of the previously described stented valve was manufactured in a similar way and with similar materials, but including several holes cut into the jugular tissue in the spaces between the stent wires. This design, created to correct the coronary blockage of the previous concept, allowed coronary flow through the stent, but the problem of early migration was still present.

One of the latest concepts in percutaneous aortic valves was designed to correct both of the problems present in the previously discussed concepts. This catheter delivered valve employed the “sandwich concept”: two concentric stents, one containing the attached jugular segment and the other surrounding the stented valve, embrace the native leaflets of the aortic valve. The diameters of the stents are calculated to match at their expanded form; this allowed them to grab the leaflets and leave some space for coronary flow between the device and the aortic sinus.

The peripheral stent is self expandable, shorter in length and can be released before the stented valve. The deployment is done in two stages, and the problem of early migration is addressed by holding the natural leaflets between the two stents.

Although this design has shown to give an acceptable short term solution to the problem of sudden migration, and obstruction of coronary flow, the amount of time the device will remain in its position is still uncertain.

The three CBHV concepts described above were used in an animal study related by Boudjemline 2002. In this study, the percutaneous implantation of these devices was performed in a group of twelve lambs so each prototype was tested in four different animals.

Another study, the first human case, was described by Cribier 2002. In this study, a more compact prototype with a stainless steel stent and leaflets made from bovine pericardium was deployed in a 57 year-old man with calcific aortic stenosis.

Both studies (Boudjemline 2002 and Cribier 2002) revealed that although the devices and procedures are still in the developmental phase, the percutaneous implantation of prosthetic heart valves was possible without previous removal of the diseased valve.

Two years after the completion of the first human case, Cribier 2004 described the experiences obtained from the implantation of CBHVs in six end-stage inoperable patients with calcific aortic stenosis. This study used an improved version of the device used in Cribier 2002. The CBHV was still made of stainless steel stents but with three equine pericardial leaflets.

The CBHV device was successfully deployed in all six cases described in the research, but early migration of one of them proved the device to be dependent on calcified tissue to reach reliable levels of attachment. In vitro studies on these devices have shown that they can run for 200 million cycles (5 years), but in vivo experiments with these devices are not likely to reveal the long term effects of the technology since clinical trials are restricted to end-stage patients.

The main advantage of a CBHV is that it could be implanted without major surgery, but one of the practical issues of the existing catheter-based valve technology, or at least in existing concepts, is that durability of existing designs is rather limited, and that the limited durability is because of a trade off between of maximizing the contraction of the device by using the least amount of material and maximizing durability by using more and stronger material.

### SUMMARY

The Catheter Based Heart Valve (CBHV) described herein is a device that replaces a non functional, natural heart valve. The CBHV significantly reduces the invasiveness of the implantation procedure by being inserted with a catheter as opposed to open heart surgery. Additionally, the CBHV is coated with a biocompatible material to reduce the thrombogenic effects and to increase durability of the CBHV.

A functional prototype is described that has a 19 mm diameter capable of being contracted to 7.3 mm. Contraction capabilities of this prototype allow its deployment via catheter to offer a less invasive alternative among heart valve disease treatments.

The CBHV includes a stent and two or more polymer leaflets sewn to the stent. The stent is a wire assembly coated with Polystyrene-Polyisobutylene-Polystyrene (SIBS). The leaflets are made from a polyester weave as a core material and are coated with SIBS before being sewn to the stent. Other biocompatible materials may be used, such as stainless steel, Titanium, Nickel-Titanium alloys, etc.

### BRIEF DESCRIPTION OF THE DRAWINGS

Objects, features, and advantages of the present invention will become apparent upon reading the following description in conjunction with the drawing figures, in which:

FIG. 1 is a perspective view of a CBHV constructed in accordance with the teachings of the disclosure including a stent and valve leaflets;

FIG. 2 is a perspective view of the stent of FIG. 1;

FIG. 3 is a schematic representation of a stent in a vessel;

FIG. 4 is a perspective view of the leaflets of FIG. 1;

FIG. 5 is a perspective view of a tension table used to form the stent of FIG. 2;

FIG. 6 is a magnified view of the leaflet material;

FIG. 7 is a magnified view of the leaflet material of FIG. 6 after coating with a biocompatible material;

FIGS. 8a-d are schematic representations of two leaflet configurations;

FIGS. 9a and b are side views of a portion of the stent of FIG. 1;

FIGS. 10a and b are side views of a portion of a modified stent;

FIGS. 11a and b are side views of a portion of yet another modified stent;

FIGS. 12a and 12b are perspective views of the stent of FIG. 9 with the two leaflet configurations of FIG. 7;

FIGS. 13a and 13b are perspective views of the stent of FIG. 10 with the two leaflet configurations of FIG. 7;

FIGS. 14a and 14b are perspective views of the stent of FIG. 11 with the two leaflet configurations of FIG. 7;

FIG. 15 is a perspective view of the stents of FIGS. 9-11 with a first leaflet configuration and in a compressed condition;

FIG. 16 is a perspective view of the stents of FIGS. 9-11 with a second leaflet configuration and in a compressed condition;

FIG. 17 is a schematic representation of a projected area of the leaflets of FIG. 8;

FIG. 18 is a schematic representation of a projected area of the stent of FIG. 11;

FIG. 19 is a graph of contraction limits for various stent configurations;

FIG. 20 is a graph of contraction limit vs. valve diameter for various stent configurations;

FIG. 21 is a schematic comparison of a stent of FIG. 9 with and without forward migration retaining projections;

FIG. 22 is a schematic representation of various stent configurations in an aortic valve;

FIG. 23 is a graphical evaluation of various CBHV configurations;

FIG. 24 is a graphical comparison of pressure difference for various heart valve configurations;

FIG. 25 is a graphical comparison of closing volume for various heart valve configurations; and

FIG. 26 is a graphical comparison of flow leakage for various heart valve configurations.

### DETAILED DESCRIPTION OF THE DISCLOSURE

The Catheter Based Heart Valve (CBHV) includes a stent and two or more leaflets attached to the stent. The stent provides structural support for the leaflets and keeps the CBHV in place in the aortic root, while minimizing obstruction of the coronary flow.

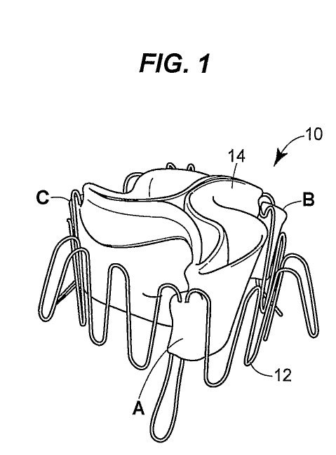

As shown in FIG. 1, the CBHV 10 includes two basic components, the stent 12 and one or more leaflets 14. The configuration shown in FIG. 1 forms an adaptable stent geometry without the need for extended sutures connecting the leaflets 14 to the stent 12. The leaflets 14 are attached to the stent 12 at three locations A, B, C. The CBHV 10 takes on a generally cylindrical shape for insertion into a vascular structure. However, the stent 12 is radially deformable and partially collapsible, due in part to the spring-like configuration of the stent 12. Thus, the stent 12 is suitable for insertion via a catheter and will form itself to the vessel shape into which the stent 12 is placed. This feature is especially beneficial for replacement of aortic valves as the aorta is generally not perfectly cylindrical in shape.

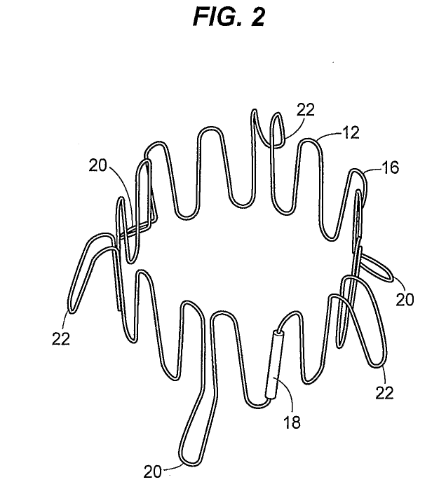

FIG. 2 shows a perspective view of the stent 12. The stent 12 is the most critical component of the CBHV 10. The stent 12 is responsible for the structural support of the leaflets 14, and the stent 12 keeps the CBHV 10 in place in the vessel. Further, the stent 12 should not obstruct coronary flow.

The stent 12 of this embodiment is constructed from a continuous piece of nitinol wire 16, the ends of which are joined with a hypodermic tube 18. The stent 12 may be made of virtually any material, however, traditional prosthetic materials (e.g., stainless steel, Titanium, Nickel-Titanium alloy, etc), or other materials that have previously been used under biological conditions and proven appropriate are generally used. The stent 12 material may be coated with SIBS, or another biocompatible coating to further enhance the biocompatibility of the CBHV 10. In one embodiment, the stent 12 has an expanded diameter of approximately 24 mm and a length of approximately 18 mm. This embodiment also has a contracted diameter of approximately 8 mm or less, thus providing a general expansion-contraction ratio of approximately 3:1. However, acceptable ranges for the expanded diameter are approximately 18 mm to approximately 27 mm; acceptable ranges for the contracted diameter are approximately 6 mm to approximately 9 mm; and acceptable lengths for the stent 12 are from approximately 12 mm to approximately 24 mm. These dimensions allow the insertion of the CBHV 10 via a catheter while still allowing the CBHV 10 to adequately cover the size of a natural leaflet.

Known catheter insertable valves generally suffer from either early migration or coronary blockage. To address the problem of early migration, the stent 12 includes forward migration retainers 20 and backflow migration retainers 22. As shown below, the forward migration retainers 20 prevent migration of the CBHV 10 in the direction of flow, while the backflow migration retainers 22 prevent migration of the CBHV 10 opposite the direction of flow, while also providing separation between the natural leaflets and the vascular wall.

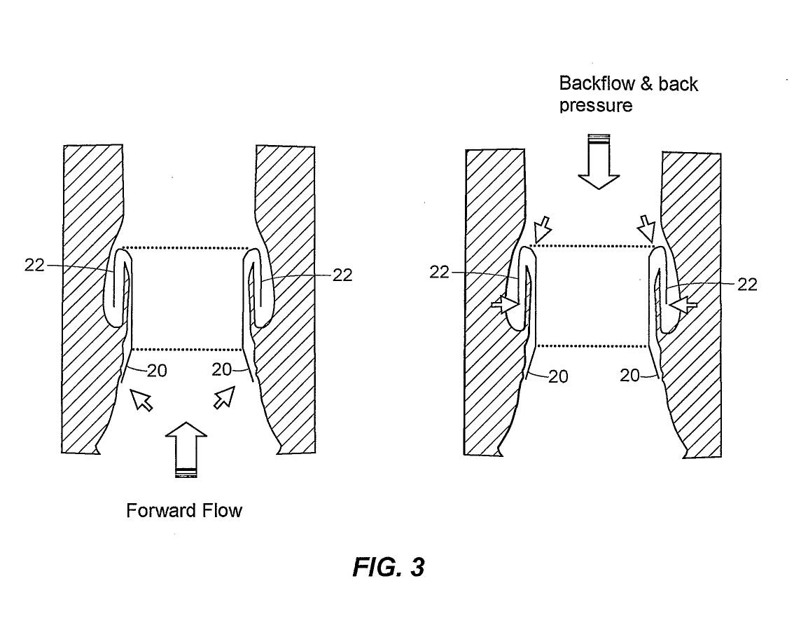

Schematics of the prototype of the CBHV are shown in FIG. 3. The left side of FIG. 3 shows the orientation of the forward migration retainers 20 against the valvular root. The right side of FIG. 3 shows the dual function of the backflow migration retainers 22 wrapping around the natural leaflets.

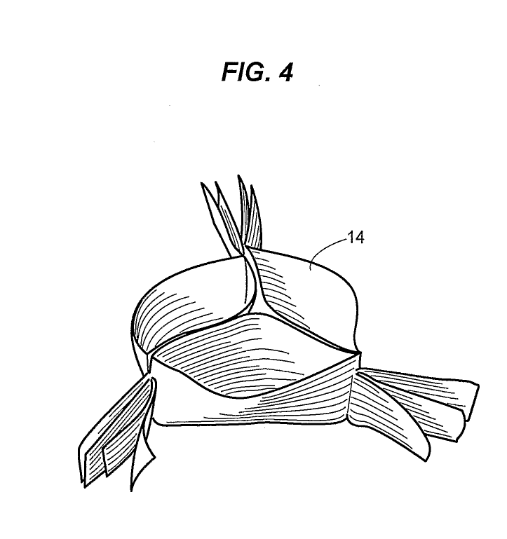

FIG. 4 shows a perspective view of the leaflets 14. The leaflets 14 are made from a woven fabric material such as a DACRON® mesh and coated with SIBS. However, other materials are acceptable, such as, polyester and polypropylene. These materials in combination with the SIBS coating have generally proven to reduce the risk of thrombi formation and thus the need for anticoagulant therapy. Initially, a sheet of DACRON® is extended and fixed over a drying plate. Next a solution of SIBS is poured and let to dry for several hours to cover the DACRON® mesh. Once dry, the DACRON® sheets are folded and sutured 24 together to create a leaflet group.

Each leaflet 14 is both peripherally and centrally coaptable. This feature allows the leaflet to have an adaptable geometry, especially peripherally and this adaptable geometry allows the leaflet 14 to be attached to the stent 12 with fewer sutures. The leaflet 14 provides a laminar flow across the leaflet when subjected to fluid flow having a viscosity similar to that of human blood. In other words, the Reynolds number of blood flowing across the leaflet 14 is less than approximately 2000. Additionally, the woven fabric material of the leaflet 14 is very durable, capable of performing more than approximately 600 million cycles before failure. Additionally, the leaflet 14 exhibits a backflow leakage of less than approximately 5%, and a backflow volume required to close of less than 2.5% of stroke volume when the leaflet 14 is used in a replacement heart valve.

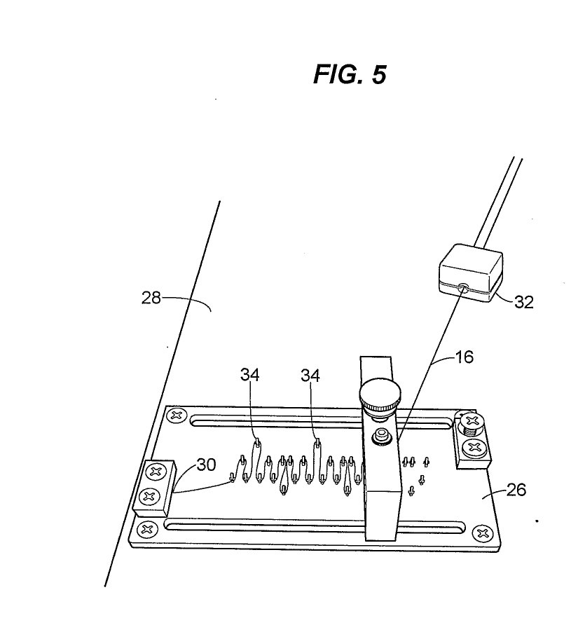

FIG. 5 shows a stent plate 26 attached to a tension table 28. In forming the stent 12, a piece of nitinol wire 16 is attached to the stent plate 26 at one end 30 and a tensor 32 at the other end. The wire 16 is stretched along a path determined by a plurality of pins 34, thus creating a geometry of the stent 12. Once the wire 16 is stretched, the stent plate 26 and wire 16 may be thermally treated to set the shape of the wire 16. One method of thermal treatment involves subjecting the wire 16 to temperatures above 500 degrees C., for a period in excess of 15 minutes. Next, a second plate (not shown) is used to form the forward migration retainers 20 and backflow migration retainers 22. A second thermal treatment may be performed to fix the shape of the forward migration retainers 20 and backflow migration retainers 22. The stent 14 may then be bent into a roughly cylindrical shape where the ends of the wire 16 are held together with a hypodermic tube 18.

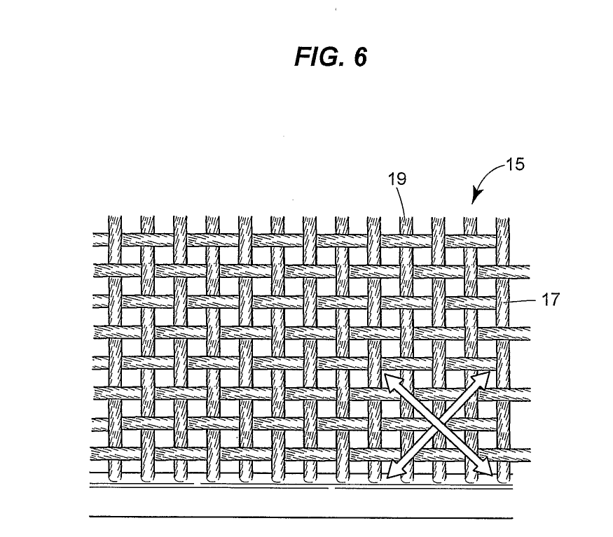

FIG. 6 shows a magnified view of a polyester fabric used to construct the leaflets 14. Generally, the leaflets may be constructed from suitable materials such as, DACRON®, Polyester and Polypropylene. Generally, the material should have a thickness of less than 280 microns so to not limit contraction of the CBHV 10 during insertion. A tradeoff exists, however, because thinner fabrics, while enhancing contraction, sacrifice durability. Although all thin polyester fabrics are suitable for the leaflets 14, weave pattern can significantly increase or reduce strength and durability of the leaflets 14. The example material shown in FIG. 6 is a polyester fabric made in a 15% dilution. The polyester fabric is made with a square thread weave pattern 15. This weave pattern 15 is strongest along orthogonal directions 17, 19 corresponding to the threads, while weakest at 45 degree angles from the threads (shown by the arrows in FIG. 6). The material of FIG. 6 has a mean fabric thickness of approximately 116 micrometers.

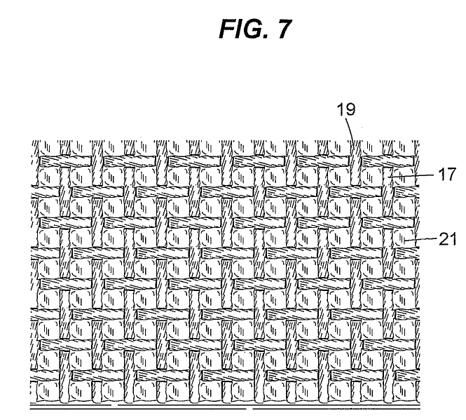

The material may be coated with SIBS and allowed to dry for 12 hours at 80 degrees C. The result of a 10 ml solution of SIBS is shown in FIG. 7. The SIBS coating generally coats the threads and generally fills in the gaps 21 between the threads. Desirably, the thinnest material with the highest quality of coating is obtained for the leaflets. However, these two design criteria operate opposite one another. For example, higher quality coatings generally thicken the material, while a thinner material necessarily has less coating, and thus a lower quality coating. Experimental results determined that a 20 ml solution of SIBS struck a balance between high quality coating and the thickness of the material.

Finally, the leaflets 14 are sewn or otherwise attached to the stent 12 and the entire CBHV 10 is coated with a SIBS film to further enhance biocompatibility (see FIG. 1).

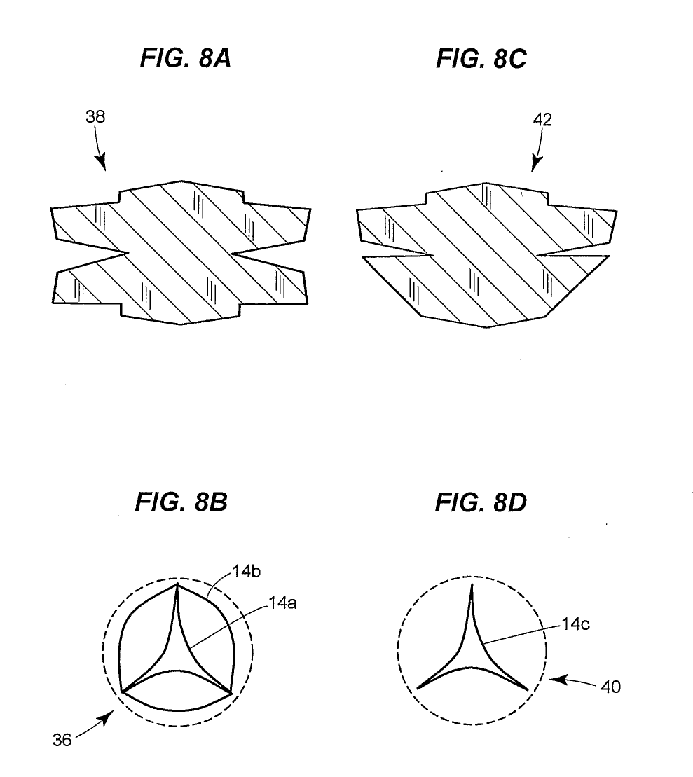

FIGS. 8a-d show two leaflet 14 configurations. FIGS. 8a and 8b show a double coaptation leaflet 36 and the planar pattern 38 from which the double coaptation leaflet 36 is formed. FIGS. 8c and 8d show a central coaptation leaflet 40 and a planar pattern 42 from which the central coaptation leaflet 40 may be formed. Both central coaptation and double coaptation leaflets may be formed from planar geometries and similar manufacturing techniques. Each of the planar patterns 38, 42 of FIGS. 8a and 8c represents one leaflet. Three such leaflets may be used for each CBHV 10. The diagonal lines shown in the planar patterns 38, 42 represent an orthogonal orientation of the threads of the material. This orientation mimics the mechanical properties of natural leaflets. Natural leaflets have a higher elasticity along lines of coaptation and lower elasticity along the flow direction. This arrangement facilitates complete coaptation and strength against pressure gradients. The thread orientation shown in FIGS. 8a and 8c gives the leaflets 14 more elastic properties along the coaptation lines and stiffer properties in directions partially aligned with the flow.

As seen in FIGS. 8a and 8b, the double coaptation leaflet 36 is formed from a single sheet of material that is folded into two plies 14a and 14b. A first ply 14a coapts centrally with other leaflet 14 plies and a second ply 14b coapts peripherally with the stent 12 or vascular wall. The fold of the centrally coaptable leaflet 14 is oriented upstream from the free ends of the two plies 14a and 14b, in a direction of blood flow.

As seen in FIGS. 8c and 8d, the central coaptaion leaflet 40 is also formed from a single sheet of material. However, the central coaptaion leaflet 40 is not folded and remains a single ply 14c. The single ply 14c coapts both peripherally and centrally. The peripheral coapation occurring at one end of the single ply 14c and the central coaptaiton occurring at the other end of the single ply 14c. One advantage of the single ply 14c is that the single ply 14c is contractable to a smaller diameter because the single ply 14 uses less material that the double ply 14a, 14b of the double coaptation leaflet shown in FIGS. 8a and 8b.

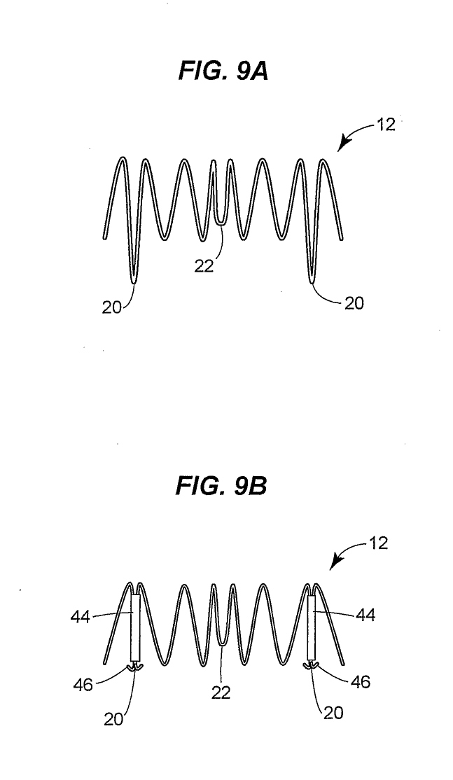

FIGS. 9a and 9b show a planar representation of a first embodiment of a stent 12 constructed in accordance with the teachings of the disclosure. This embodiment is called the “Pioneer” stent. The stent 12 of FIGS. 9a and 9b includes backflow migration retainers 22 and forward migration retainers 20. The stent 12 of FIG. 9a includes forward migration retainers 20 that are bent loops of wire. The stent 12 of FIG. 9b includes forward migration retainers 20 that have the loops of wire brought together with a sheath 44, and the ends of the loop are cut and formed into hooks 46. The hooks 46 are added to enhance attachment of the stent 12 to the vessel wall. The planar representations shown in FIGS. 9aand 9b represent one third of a total stent 12 with the pattern shown repeating around the circumference of the stent 12. This design has a relatively high number of wire turns which limits the contraction of the stent 12 somewhat. The relatively high number of turns also increases the material required for the stent 12.

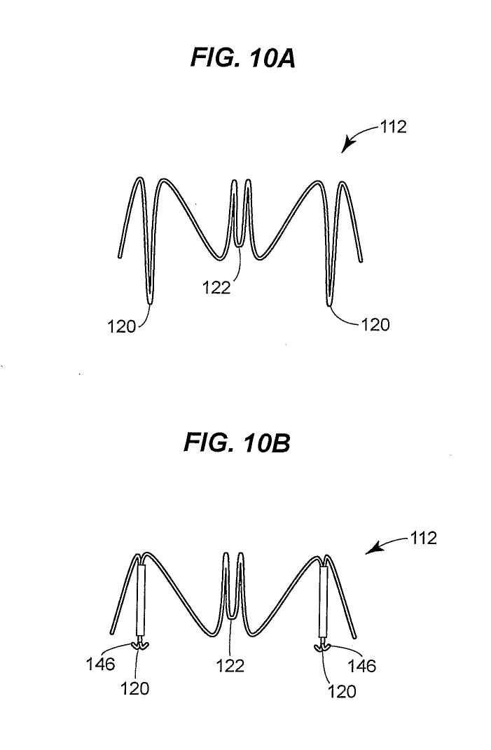

FIGS. 10a and 10b show a planar representation of a second embodiment of a stent 112. This embodiment is called the “Simplified” stent. This stent 112, like the embodiment of FIGS. 9a and 9b, includes backflow migration retainers 122 and forward migration retainers 120. Also, like the embodiment of FIGS. 9a and 9b, the stent 112 of FIG. 10a uses bent loops of wire to form the migration retainers 120, 122 and the stent 112 of FIG. 10b modifies the forward migration retainers 120 to include hooks 146. As is seen in FIGS. 10a and 10b, this second embodiment includes fewer wire turns and thus requires less wire material. Furthermore, the reduced wire turns enhance the contraction of the stent 112, thus potentially allowing a smaller diameter catheter to be used for insertion of the stent 112.

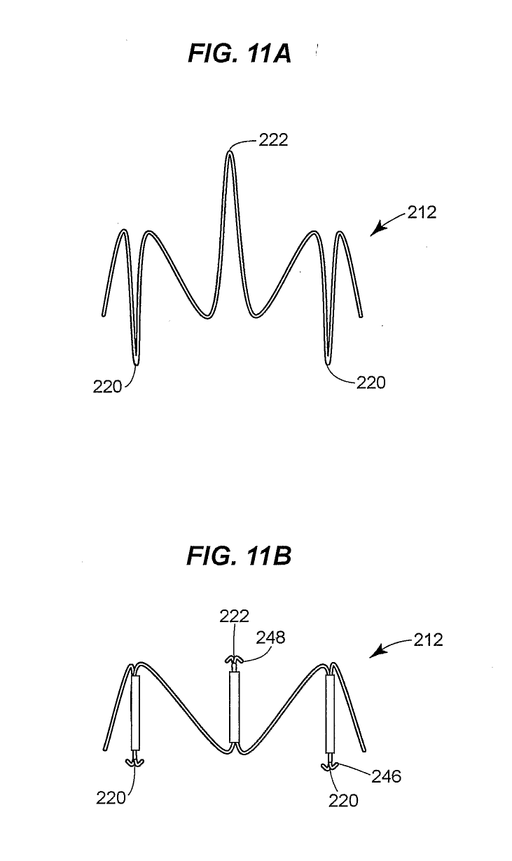

FIGS. 11a and 11b show a planar representation of a third embodiment of a stent 212. This embodiment is called the “Modified” stent. This stent 212, like those of the embodiments of FIGS. 9a, 9b and 10a, 10b, includes backflow migration retainers 222 and forward migration retainers 220. Also, like those of the embodiments of FIGS. 9a, 9b and 10a, 10b, the stent 212 of FIG. 11a uses bent loops of wire to form the migration retainers 220, 222 and the stent 212 of FIG. 11b includes modifications to the forward migration retainers 220 to include hooks 246. However, unlike previous embodiments, the embodiment of FIG. 11b also includes modifications to the backflow migration retainers 222 to include hooks 248. The embodiment shown in FIG. 11b eliminates the additional turns required to form the backflow migration retainers 222 of the embodiment shown in FIG. 10a, 10b. As a result, the third embodiment of the stent 212, shown in FIG. 11b has the greatest contractive ability of all three embodiments.

A nomenclature system using combinations of single-letter feature designations was adopted for each prototype. In this system, for example, every prototype that contained Double Coaptation Leaflets included the letter “D” in their reference name. So for a prototype that used a Modified stent with Double Coaptation Leaflets and Forward Flow Hooks the abbreviation “MDF” was used to name it. See Table 1 below for the full one-letter code used to name the prototypes.

TABLE 1

| | | ONE |
|---|---|---|
| | | LETTER |
| | FEATURES | CODE |
| Stent Types | Pioneer Stent | P |
| | Simplified Stent | S |
| | Modified Stent | M |
| Leaflet Types | Central Coaptation Leaflets | C |
| | Double Coaptation Leaflets | D |
| Attachment Mechanism | Forward Flow Hooks | F |
| | Backflow &Forward Hooks | B |
| | No Hooks | N |

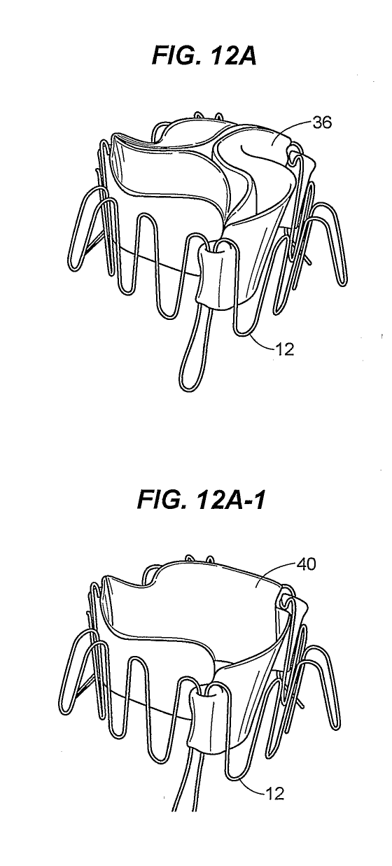

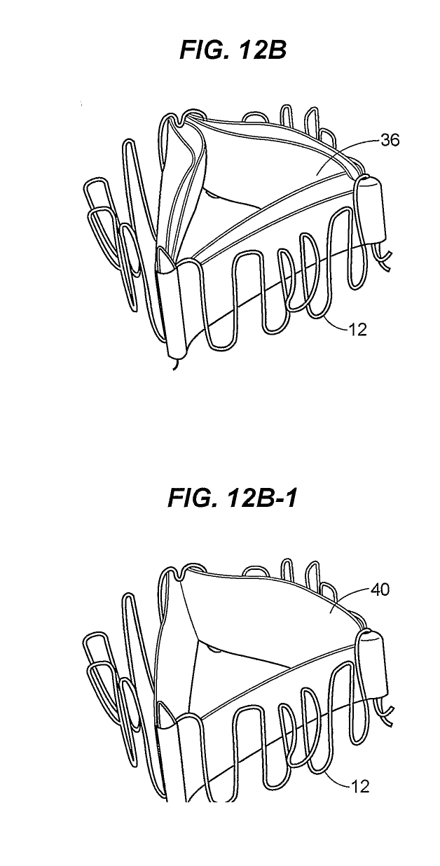

FIGS. 12a and 12b show the Pioneer stent 12 of FIGS. 9a, 9b, both with and without hooks and having either a double coaptation 36 or a central coaptation 40 leaflet. Specifically, the stent 12 of FIG. 12a is the Pioneer stent 12 of FIG. 9a, joined with a double coaptation leaflet 36 (FIG. 12a) (PDN) and a central coaptation leaflet 40 (FIG. 12a-1) (PCN). Likewise, the stent 12 of FIG. 12b is the Pioneer stent 12 of FIG. 9b, joined with a double coaptation leaflet 36 (FIG. 12b) (PDF) and a central coaptation leaflet 40 (FIG. 12b-1 (PCF).

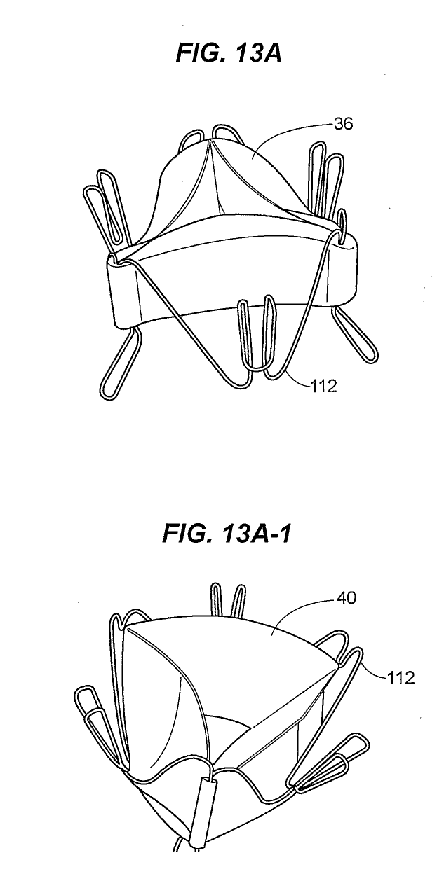

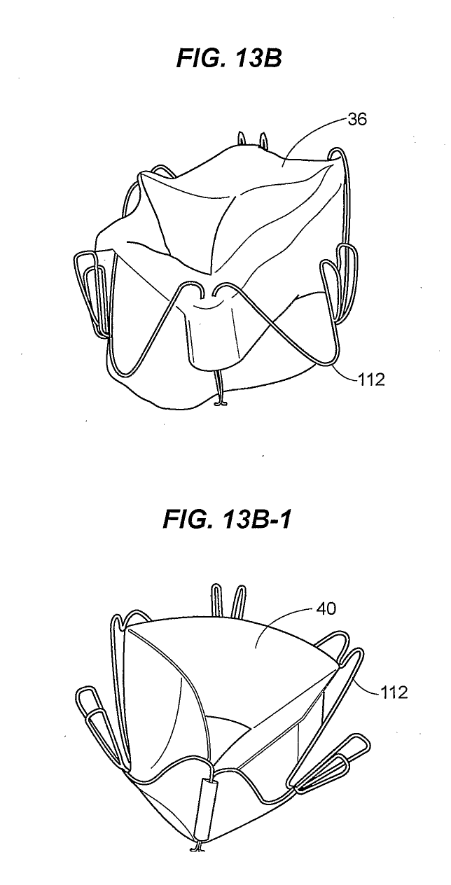

Similarly, FIGS. 13a and 13b show the Simplified stent 112 of FIGS. 10a, 10b, both with and without hooks and having either a double coaptation 36 or a central coaptation 40 leaflet. Specifically, the stent 112 of FIG. 13a is the Simplified stent 112 of FIG. 10a, joined with a double coaptation leaflet 36 (FIG. 13a) (SDN) and a central coaptation leaflet 40 (13a-1) (SCN). Likewise, the stent 112 of FIG. 13b is the Simplified stent 112 of FIG. 10b, joined with a double coaptation leaflet 36 (FIG. 13b)(SDF) and a central coaptation leaflet 40 (FIG. 13b-1) (SCF).

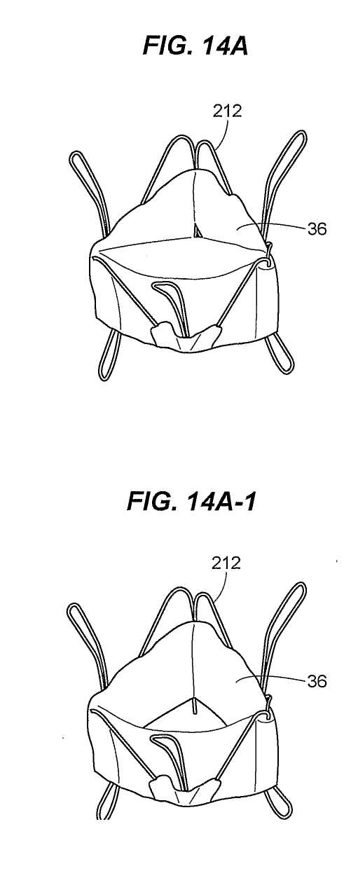

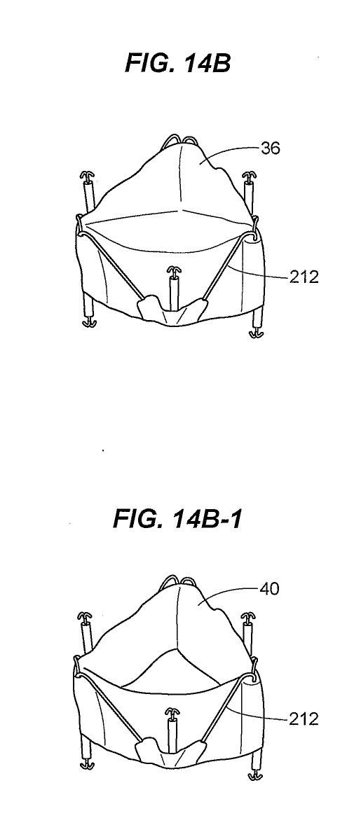

Additionally, FIGS. 14a and 14b show the Modified stent 212 of FIGS. 11a, 11b, both with and without hooks and having either a double coaptation or a central coaptation leaflet. Specifically, the stent 212 of FIG. 14a is the Modified stent 212 of FIG. 11a, joined with a central coaptation leaflet 36 (FIG. 14a) (MCN) and a double coaptation leaflet 40 (FIG. 14a-1) (MDN). Likewise, the stent 212 of FIG. 14b is the Modified stent 212 of FIG. 11b, joined with a double coaptation leaflet 36 (FIG. 14b) (MDB) and a central coaptation leaflet 40 (FIG. 14b-1) (MCB).

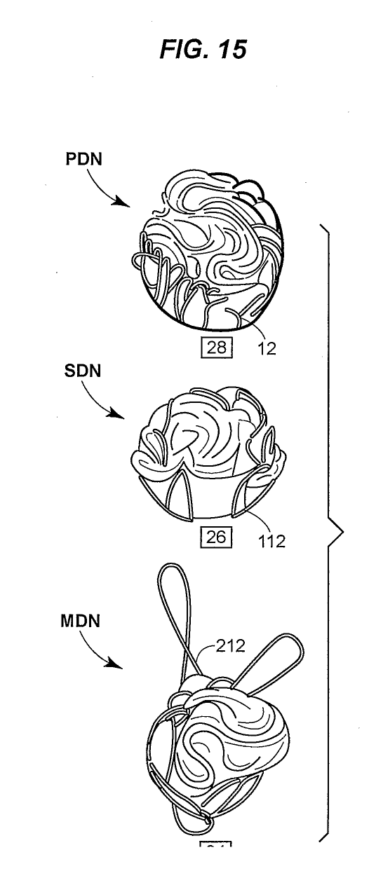

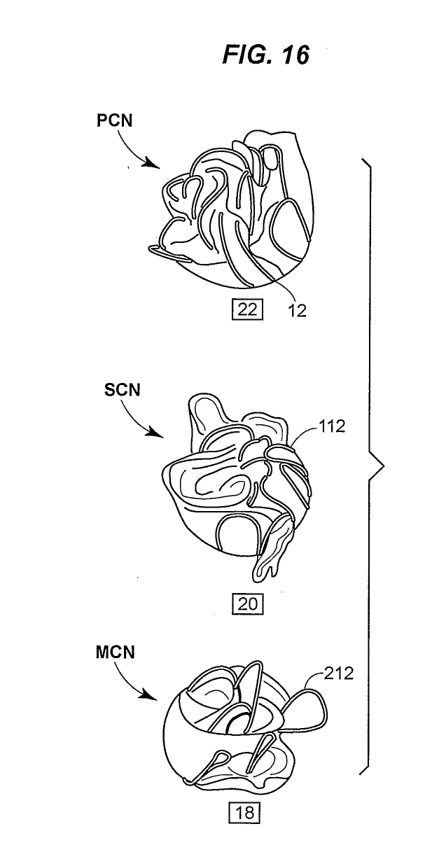

Maximum contraction is a primary factor in determining the suitability of a CBHV 10. The smaller the CBHV 10 can contract, the smaller the diameter of a catheter is necessary for delivery of the CBHV 10 to the installation site. FIGS. 15 and 16 show the various embodiments of FIGS. 12a, 12b to 14a, 14b, in a contracted state and disposed inside circular gages for catheter diameters.

FIG. 15 shows additional versions of the CBHV 10, which include double coaptation leaflets 36. The PDN is shown disposed in a 28 gage diameter hole, the SDN is shown disposed in a 26 gage diameter hole and the MDN is shown disposed in a 24 gage diameter hole.

FIG. 16 shows further additional versions of the CBHV 10, which include central coaptation leaflets 40. The PCN is shown disposed in a 22 gage diameter hole, the SCN is shown disposed in a 20 gage diameter hole and the MCN is shown disposed in a 18 gage diameter hole.

Thus, minimum contraction diameter is shown to be a function both of stent design and leaflet type. In general, the Modified stent 212 of FIGS. 11a, 11b contracts to the smallest diameter while the Pioneer stent 14 of FIGS. 9a, 9b contracts to the largest diameter. Likewise, the central coaptation leaflets 40 of FIG. 8c, 8d, generally produce a smaller contraction diameter than double coaptation leaflets 36 of FIG. 8a, 8b. Of these two factors, leaflet configuration was more critical to designing a CBHV 10 having a minimum contracted diameter. Changes in stent design affected contracted diameter by approximately one unit, while leaflet configuration affected contracted design by approximately six units.

As a result, a mathematical formula was derived that expresses Minimum Circular Area (MCA) of a CBHV 10 as a function of Projected Area of the Leaflets (PAL) and Projected Area of the Stent (PAS). While the MCA of a CBHV 10 may aid in selection of a particular type of CBHV 10, the CBHV 10 should not be actually contracted to its MCA because of undesirable effects on the leaflets 14. Such undesirable effects include wrinkles in the leaflet, improper folding of the leaflet and entanglement of some sections of the stent wire.

The MCA may be expressed as:

$$M C A = \frac{π \times {( {C}_{l} )}^{2}}{36} \qquad Equation 1$$

Where MCA is the rearranged expression for the area of a circle in which Cₗ is the diameter measured in French Scale that represents the Contraction Limit of the device.

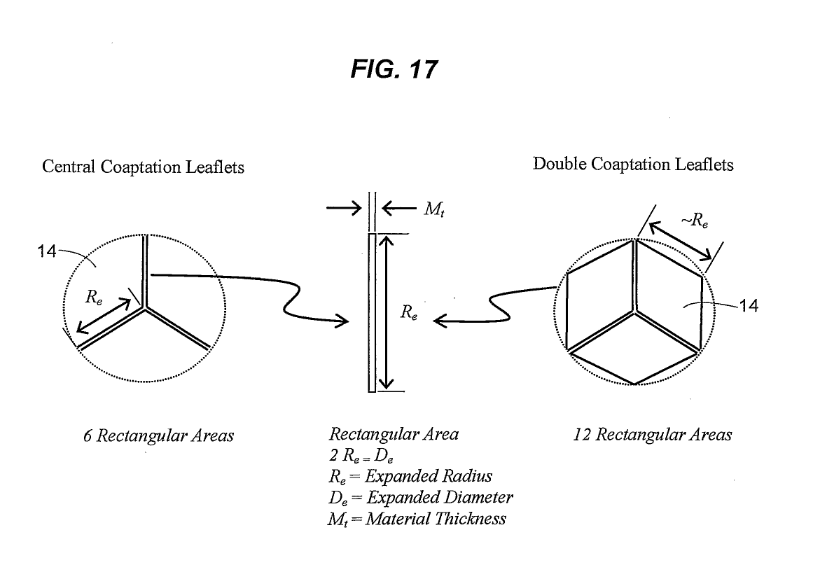

The following term of the relationship is the Projected Area of the Leaflets (PAL). This PAL is composed by the summation of the rectangular areas formed by the top edge of the leaflets 14. See FIG. 17. Notice that leaflet 14 dimensions oriented along a cylindrical axis of the valve are not considered to have a significant effect in the contraction of the leaflets.

Thus:

PAL=iᵥ×Mₜ×Dₑ Equation 2

Equation 2 is the result of the summation of all the rectangular areas that belong to a particular type of leaflet. Using the fact that 2Rₑ=Dₑ, the Projected Leaflet Area can turn into two expressions, corresponding to each leaflet type: PAL=3×Mₜ×Dₑ for Central coaptation leaflets, and PAL=6×Mₜ×Dₑ for Double Coaptation Leaflets.

The numeric coefficients in the last two expressions represent the values for iᵥ, which is the Valve Index. Mₜ and Dₑ are respectively the material thickness and the diameter of the expanded device both in millimeters.

The PAS, unlike the PAL, was not made dependent on the expanded diameter of the stent (without leaflets attached); that is explained by a simple practical reason: all prototypes, regardless of its functional diameter, were manufactured with the same stent size, but even though all the prototypes were manufactured using a single stent size, it was possible to create valves with different finctional diameters that covered all the sizes used in human applications by modifying the dimensions of the leaflet patterns to match the size required by its functional diameter.

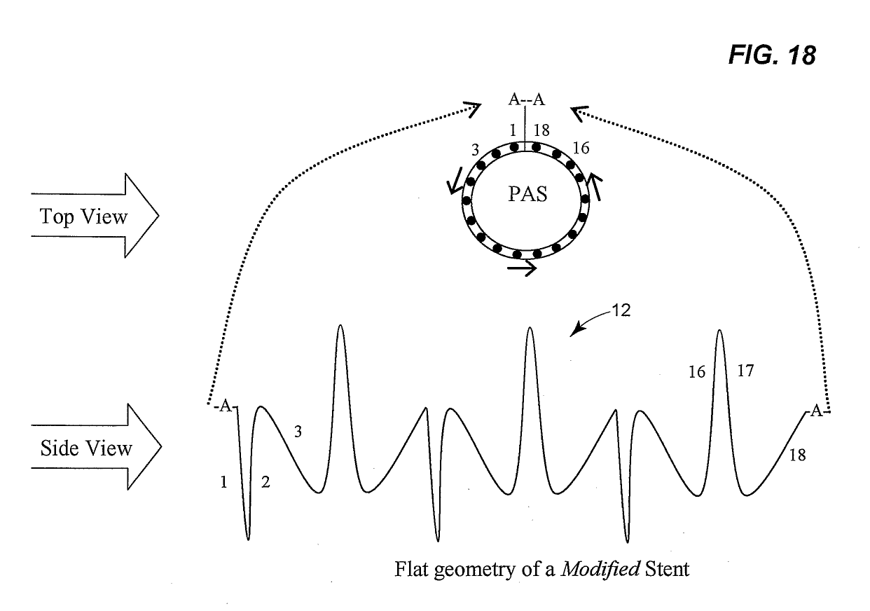

Since the expanded diameter was not a variable, additional factors were responsible for determining The Projected Area of the Stent. In a similar fashion to the Projected Area of the Leaflets, PAS was determined from the circular cross sectional areas of the stent wire that were visible from the top view. In other words, PAS was the product of the cross sectional area of the stent wire by the number of times this area was present in the contracted valve. See FIG. 18 that depicts details on the measurement of the Projected Area of the Stent (PAS). Numbers on side and top views number some of the 18 cross sectional areas that compose a Modified Stent. Points labeled with the letter A on the side view correspond to the same point in the closed-loop form of the stent.

Thus:

$$P A S = \frac{{i}_{s} \times π \times {D}_{w}^{2}}{4} \qquad Equation 3$$

In Equation 3, iₛ represents the Stent Index, and D_{w} is the wire diameter in millimeters. The Stent Index is a variable introduced to account for the difference in projected areas between the three types of stents. It was calculated based on the Modified Type of stent since its geometry contained the basic features present in all stents.

The Modified type of stent without hooks (used in prototypes MCN or MDN) which contained a total of 18 projected wire areas: therefore an i_{s-Modified}=18 was used as the reference value to estimate the other two values for i_{s-Simplifed} and i_{s-Pioneer}.

After comparing actual measurements with the calculations, it was determined that the three stent models used in this project were related according to the following relationships:

i_{s-Modified}=18

i_{s-simplified}=2×i_{s-Modified}=36

i_{s-Pioneer}=3×i_{s-Modified}=54

The complete equation for the Contraction Limit includes one last coefficient: the Packing Factor (P_{f}).

The addition of the Projected Areas of the Leaflets and the Stent (PAL+PAS) is actually half the value of the actual Minimum Circular Area (MCA). To compensate the inequality caused by the omission of so called “unmeasurable” effects, a Packing Factor equal to 2 (P_{f}=2) is included.

MCA=P_{f}(PAL+PAS) Equation 4

Finally, by substituting Equations 1, 2 and 3 in Equation 4 and solving for the Contraction Limit (Cₗ) the following equation is obtained.

$${C}_{l} = 6 \sqrt{{P}_{f} ( \frac{{i}_{s} \times {D}_{w}^{2}}{4} + \frac{{i}_{v} \times {M}_{t} \times {D}_{e}}{π} )} \qquad Equation 5$$

The Contraction Limit, despite being a fairly reliable tool, may show some discrepancies with the actual contracted diameter of Modified Stents with hooks (MCB and MDB). To correct for the increase in diameter in these models, a constant value of 1.5 F should be added to the calculated value for Cₗ.

To continue, the Contraction Limit of each device was calculated using Equation 5 with the input variables shown in Table 2.

TABLE 2
Values for the input variables used in the calculation
of the Contraction Limits of the prototype CBHVs.

| CBHV Characteristics | Variable Name | Measurement | Units |
|---|---|---|---|
| Expanded Diameter | Dₑ | 19 | mm |
| Wire Diameter | D_{w} | 0.4826 | mm |
| Material Thickness | Mₜ | 0.197 | mm |

| Indices | Variable Name | Leaflet/Stent Type | Value |
|---|---|---|---|
| Packing Factor | Pf | All types | 2 |
| Valve | iv | Central | 3 |
| | | Double | 6 |
| Stent | is | Pioneer | 54 |
| | | Simplified | 36 |
| | | Modified | 18 |

After the computation of numerical values representing the Contraction Limits, the following results were obtained. See Table 3.

TABLE 3
Contraction Limits for the different types of Stent-Leaflet configurations.
Contraction Limits in French Scale
19 mm Valve Diameter

LEAFLET TYPE

| | | Central | Double |
|---|---|---|---|
| STENT TYPE | Indices | 3 | 6 |
| Pioneer | 54 | 21.99 | 27.22 |
| | | PCN - PCF | PDN - PDF |
| Simplified | 36 | 20.20 | 25.79 |
| | | SCN - SCF | SDN - SDF |
| Modified | 18 | 18.24 MCN | 24.29 MDN |
| | | 19.74 MCB | 25.79 MDB |

Values measured in French scale. Not including models MCB and MDB.

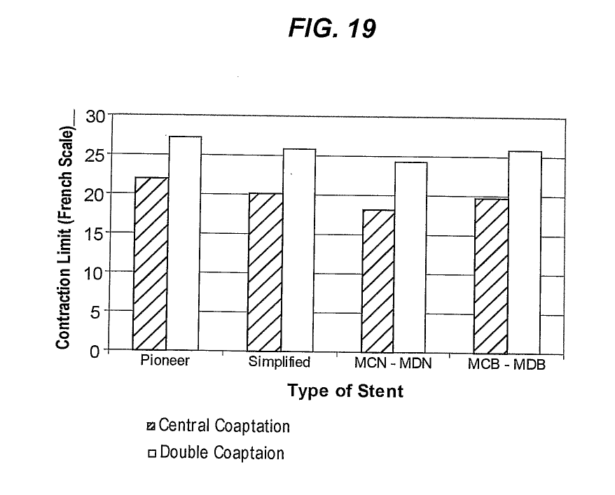

Using the results above, it can be seen how the presence of double coaptation leaflets had an effect in all the contracted sizes of the prototypes. In general, all the prototypes that incorporated leaflets of central coaptation were able to reduce the contraction limit of their corresponding stent model by at least 5 French units. See FIG. 19.

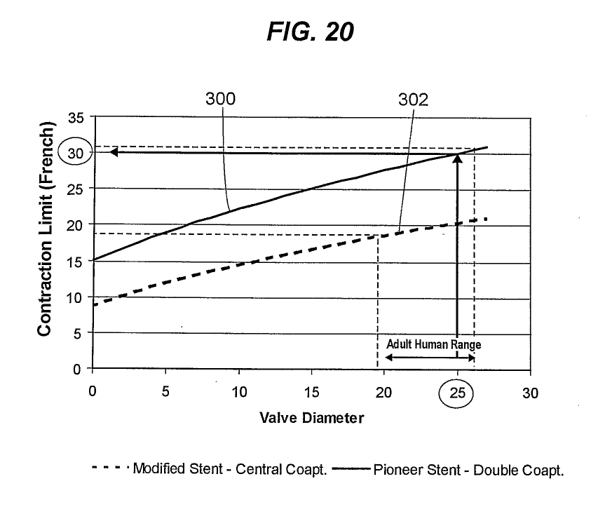

By observing the highest and lowest values of the contraction limits shown in Table 3, it can be seen that the Pioneer models with double coaptation (PDN or PDF) had the highest Contraction Limits (27.22 F); while, prototypes with MCN features had the lowest (18.24 F). These two extreme cases can be used to analyze the range of design possibilities in a different and useful manner: using Equation 5 with all the coefficients and indices set according to Table 1 for each one of the cases and leaving the Contraction Limit as a function of the Expanded Diameter (Dₑ), two curves 300, 302 representing all the Contraction Limits can be obtained for both cases. See FIG. 20.

These curves have great applicability in the design of different sizes of CBHV. For example, if a valve type PDN having a functional diameter of 25 mm were selected to be used in a hypothetical in-vivo study, it would be possible to know before its manufacture that its contraction capabilities would require that the deployment system as well as the vessel anatomy allow diameters greater than 30 F.

The ability of the devices to adapt to the geometry of the aortic root depends on the expansive force of the stent. Measurements of the expansive force of the stent models were made, but manual contraction of the devices offered a simplified method for estimating and comparing such force among the stents.

Using manual gauging, it was determined that the level of expansive force was the lowest in the Pioneer models and the highest in the Modified ones; this information added to observations on the peripheral contact of the stent with the aortic root was used to evaluate the adaptability of a stent to the anatomical features.

Pioneer stents, with the weakest expansive force, were observed to have less contact with the aortic walls. This situation was frequently encountered in areas close to the backflow retainers. Different from Pioneer stents, Simplified models had higher expansive force; this helped them to adapt more tightly to the anatomy of the aorta. In general, Simplified stents were observed to have good geometry adaptation even in zones containing backflow retainers.

Modified stents showed the best geometry adaptation of all prototypes. Two different situations were present in this group of stents: one for the stents without hooks and the other for the stents with hooks. Modified stents without hooks showed a very good level of adaptation to the anatomy of the vessel. For the case of modified stents with hooks, the levels of geometry adaptation were also very good. Contact of the stent with the aortic wall was accomplished in all its periphery.

Conclusions on Mitral Valve Interference

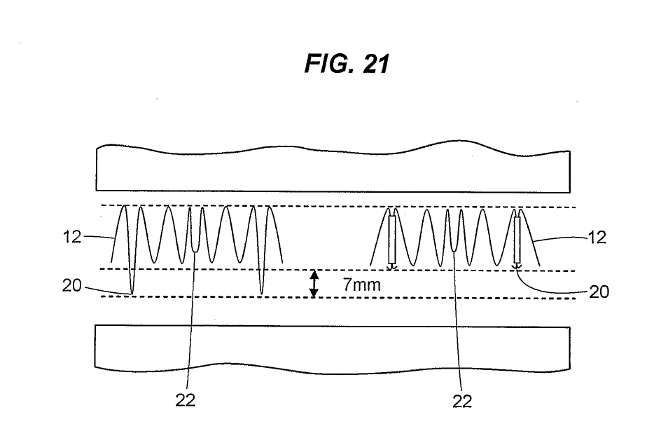

One of the constraints that the heart anatomy poses on the design of any prosthetic heart valve for the aortic position is the proximity of one of the mitral leaflets to the aortic root. The distance from the bottom of the aortic leaflets to the mitral leaflets is usually not greater than 3 mm, which limits the room for attachment of the upstream region of the devices. Depending on the stent model, all the tested devices had either hooks or stent projections that were intended to enhance the attachment of the device. In the case of the CBHVs with projections, the length of the upstream region of the devices was increased by about 7 mm. See FIG. 21 showing the length of two sample stents. The stent on the left is 7 mm longer than the stent on the right. The presence of projections or forward flow retainers increased the length of prototypes without hooks.

The difference in length among the stent models was observed to be directly related with the degree of mitral valve interference. All stent models that did not include hooks in their design, were observed to make direct contact with the mitral leaflet; this lead to the conclusion that stents with shorter profile were less likely to interfere with the mitral leaflets; this is true only if they have been accurately positioned and if the attachment is good enough to avoid any kind of migration towards the ventricular side of the valve.

Conclusions on Attachment

Similar to the degree of geometry adaptation, the attachment of the devices was also observed to be dependent on the expansive force of the device. For all different models of stents, the higher the expansive force the better the attachment of the device to the aortic walls. In devices that did not have hooks or backflow retainers, the attachment was essentially determined by the expansive force of its stent. The higher the force that the stent made against the aortic walls, the higher was the friction force that was created to prevent migration.

In other devices with backflow retainers covering the leaflets of the natural valve part of the attachment of the device was obtained by the physical interaction of the retainers with the natural leaflets. This interaction prevented migration of the device into the ventricle, but did not offer noticeable attachment in the direction of the flow.

The main conclusion regarding attachment was that the presence of hooks in the stent made a difference during its extraction, and such difference was more accentuated in stents with higher expansive force. Prototypes including hooks had better attachment.

Conclusions on Coronary Obstruction

Coronary obstruction and mitral valve interference are two different problems that arise from the same cause: the length of the stent. Three different situations can occur depending on the length of the stent: Coronary obstruction, mitral valve interference or both.

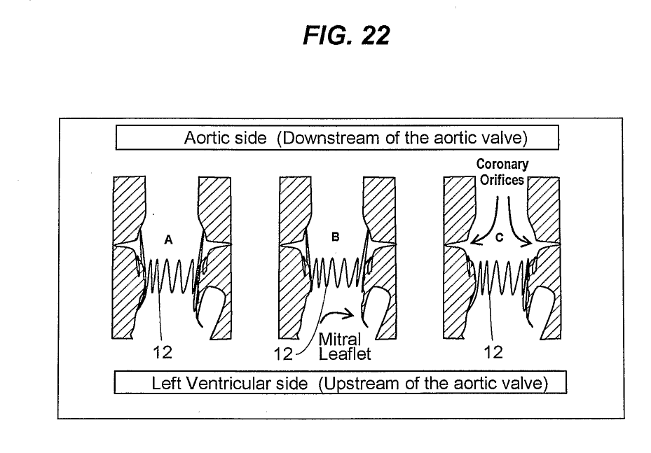

When the length of the stent inside the aortic root exceeds the distance between the coronary orifices and the mitral valve leaflet, cases of mitral valve interference and coronary obstruction are observed. The two other cases can depend on the design of the stent; if the stent is longer in the downstream region of the valve it is possible to have coronary obstruction; while if the stent is longer in the upstream region of the valve it is possible to have mitral valve obstruction. An illustration of the three situations is shown in FIG. 22. A) shows coronary and mitral valve obstruction, B) shows coronary obstruction and C) shows mitral valve interference.

From the three possible cases shown in FIG. 22, cases A and C were observed during testing. Although case B could also produce coronary obstruction, no prototypes had with such characteristics.

The prototypes that were likely to obstruct the coronary orifices were the MCN and the MDN. In both cases the projections of the stent were long enough to produce the situation depicted in case A above.

Conclusions on Leaflet Unfolding

After deployment, the expansion of the devices was expected to produce a correct configuration of the leaflets in each valve. However, for valves with leaflets of double coaptation, the deployment of the device occasionally led to incorrect unfolding of the leaflets whereas in valves with central coaptation the leaflets unfolded correctly. The explanation for the improper unfolding in the case of leaflets with double coaptation may be found in the irregular shape of the aortic root. Thus, the double coaptation configurations are better suited to use in more regularly shaped vessels. The designs of all leaflets used in the prototypes were originated from the assumption that the aortic root had a circular cross section. This assumption, although very practical in terms of design, it did not foresee the effects of irregular anatomies in leaflet configuration. Since leaflets with double coaptation had a more complex structure than the ones with central coaptation, small changes in the deployment position or in the circularity of the vessel were observed to interfere with the correct formation of the leaflets.

Hemodynamic Testing—Quantitative Session

Prior to the initiation of tests, baseline readings were recorded for the aortic, ventricular and flow transducers. The testing was done according to the flow regimes shown below in Table 4.

TABLE 4

| | Flows Regimes | | | |
|---|---|---|---|---|
| | (L/min) | | | Test Duration |
| Heart Rate (Beats/min) | Low | Moderate | High | (Seconds) |
| 50 | 2 | 4 | 6 | 72 |
| 70 | 4 | 6 | 8 | 52 |
| 90 | 6 | 8 | 10 | 40 |
| 120 | 8 | 10 | 12 | 30 |
| 150 | 10 | 12 | 14 | 24 |
| 180 | 12 | 14 | 16 | 20 |

All prototypes including the natural aortic valve were tested under the regimes shown above. The testing procedure followed a factorial design that started from the slowest cardiovascular regime (50 bpm and 2 L/min) and was gradually increased up to the extreme conditions generated at 180 bpm.

The first valve that was tested was the natural aortic valve. Readings for flow rates, aortic and ventricular pressures were used to set the ideal performance that any prosthetic valve could reach. Following the complete testing of the natural valve, three replicates of the best CBHV prototype were tested. The best CBHV prototype was selected from all qualitative tests previously done.

After the completion of the CBHV prototype's testing, a polymer valve with a rigid stent was sutured on top of the natural aortic valve. Hemodynamic measurements from this valve were recorded to be used as a control and benchmark for the performance of the CBHV prototypes.

Qualitative Tests

Qualitative assessment of the prototypes under static conditions delivered significant information that allowed determination of which of the CBHV prototypes were the most likely to be excluded from the quantitative tests, but the final decision about which one of the twelve prototypes was the best required hemodynamic observations.

In order to establish an objective basis for the comparison and screening of the prototypes, decision matrices were created from all qualitative observations collected during static and hemodynamic tests respectively; each one of the criteria used in those tests was weighed according to its importance.

In the case of the decision matrix for static tests, the most critical factors were mitral interference, coronary obstruction and attachment; these factors were all graded in a scale from zero to five while the rest of the factors, deployment difficulty, leaflet unfolding and geometry adaptation, were only graded in a scale from zero to three. Table 5 shows the actual matrix.

TABLE 5

| | STATIC TESTS | | | | | | | | | | | | | | | | | | | | | | | | |
|---|---|---|---|---|---|---|---|---|---|---|---|---|---|---|---|---|---|---|---|---|---|---|---|---|---|
| | Deployment | | | Leaflet | | | Geometry | | | | | | | | | | | | | | | | | | SUB |
| Prototype | Difficulty | | | Unfolding | | | Adaptation | | | Mitral Interference | | | | | Coronary Obstruction | | | | | Attachment | | | | | TOTALS |
| PDN | −1 | −1 | −1 | 1 | 0 | 0 | 0 | 0 | 0 | −1 | −1 | −1 | −1 | −1 | 0 | 0 | 0 | 0 | 0 | 1 | 0 | 0 | 0 | 0 | −6 |
| PCN | −1 | −1 | −1 | 1 | 1 | 1 | 0 | 0 | 0 | −1 | −1 | −1 | −1 | −1 | 0 | 0 | 0 | 0 | 0 | 1 | 0 | 0 | 0 | 0 | −4 |
| PDF | −1 | −1 | −1 | 1 | 0 | 0 | 0 | 0 | 0 | 0 | 0 | 0 | 0 | 0 | 0 | 0 | 0 | 0 | 0 | 1 | 1 | 0 | 0 | 0 | 0 |
| PCF | −1 | −1 | −1 | 1 | 1 | 1 | 0 | 0 | 0 | 0 | 0 | 0 | 0 | 0 | 0 | 0 | 0 | 0 | 0 | 1 | 1 | 0 | 0 | 0 | 2 |
| SDN | −1 | −1 | −1 | 1 | 0 | 0 | 1 | 1 | 1 | −1 | −1 | −1 | −1 | −1 | 0 | 0 | 0 | 0 | 0 | 1 | 1 | 0 | 0 | 0 | −2 |
| SCN | −1 | −1 | −1 | 1 | 1 | 1 | 1 | 1 | 1 | −1 | −1 | −1 | −1 | −1 | 0 | 0 | 0 | 0 | 0 | 1 | 1 | 0 | 0 | 0 | 0 |
| SDF | −1 | −1 | −1 | 1 | 1 | 1 | 1 | 1 | 1 | 0 | 0 | 0 | 0 | 0 | 0 | 0 | 0 | 0 | 0 | 1 | 1 | 1 | 0 | 0 | 6 |
| SCF | −1 | −1 | −1 | 1 | 0 | 0 | 1 | 1 | 1 | 0 | 0 | 0 | 0 | 0 | 0 | 0 | 0 | 0 | 0 | 1 | 1 | 1 | 0 | 0 | 4 |
| MDN | 0 | 0 | 0 | 1 | 0 | 0 | 1 | 1 | 0 | −1 | −1 | −1 | −1 | −1 | −1 | −1 | −1 | −1 | −1 | 0 | 0 | 0 | 0 | 0 | −7 |
| MCN | 0 | 0 | 0 | 1 | 1 | 1 | 1 | 1 | 0 | −1 | −1 | −1 | −1 | −1 | −1 | −1 | −1 | −1 | −1 | 0 | 0 | 0 | 0 | 0 | −5 |
| MDB | 0 | 0 | 0 | 1 | 0 | 0 | 1 | 1 | 1 | 0 | 0 | 0 | 0 | 0 | 0 | 0 | 0 | 0 | 0 | 1 | 1 | 1 | 1 | 1 | 9 |
| MCB | 0 | 0 | 0 | 1 | 1 | 1 | 1 | 1 | 1 | 0 | 0 | 0 | 0 | 0 | 0 | 0 | 0 | 0 | 0 | 1 | 1 | 1 | 1 | 1 | 11 |

The cells belonging to each criterion were filled with numerical values that quantified the differences in observations among prototypes. The sub-total for the static test was calculated by simple addition of all the numerical values given to a prototype. After comparing the total values obtained during static tests the prototype MCB obtained the highest grade followed by the prototype MDB.

To complete the screening process of the prototypes, another decision matrix was created using hemodynamic studies. The grading system was similar to the one used in the previous matrix, but the grading scales for migration and leaflet operation established from zero to eight due to their importance. Coaptation level were graded in a scale from zero to five. See Table 6.

TABLE 6

| | QUALITATIVE HEMODYNAMIC TESTS | | | | | | | | | | | | | | | | | | | | | |
|---|---|---|---|---|---|---|---|---|---|---|---|---|---|---|---|---|---|---|---|---|---|---|
| | | | | | | Migration Regime | | | | | | | | Leaflet Operating Range | | | | | | | | |
| | | | | | | 50 | | 70 | | 90 | | 120 | | 50 | | 70 | | 90 | | 120 | | SUB |
| Prototype | Coaptation Level | | | | | L | H | L | H | L | H | L | H | L | H | L | H | L | H | L | H | TOTALS |
| PDN | 1 | 0 | 0 | 0 | 0 | 1 | 1 | 1 | 1 | 1 | 1 | 1 | 0 | 0 | 0 | 0 | 0 | 0 | 0 | 0 | 0 | 8 |
| PCN | 1 | 1 | 0 | 0 | 0 | 1 | 0 | 0 | 0 | 0 | 0 | 0 | 0 | 1 | 0 | 0 | 0 | 0 | 0 | 0 | 0 | 3.5 |
| PDF | 1 | 1 | 1 | 0 | 0 | 1 | 1 | 1 | 1 | 1 | 1 | 1 | 1 | 0 | 1 | 1 | 1 | 1 | 1 | 1 | 1 | 16 |
| PCF | 1 | 1 | 0 | 0 | 0 | 1 | 1 | 1 | 1 | 0 | 0 | 0 | 0 | 0 | 0 | 1 | 1 | 0 | 0 | 0 | 0 | 8 |
| SDN | 1 | 1 | 0 | 0 | 0 | 1 | 1 | 1 | 1 | 1 | 1 | 1 | 1 | 0 | 1 | 1 | 1 | 1 | 1 | 1 | 1 | 13.5 |
| SCN | 1 | 1 | 0 | 0 | 0 | 1 | 1 | 1 | 1 | 1 | 1 | 1 | 0 | 0 | 1 | 1 | 1 | 1 | 1 | 1 | 0 | 12.5 |
| SDF | 1 | 1 | 0 | 0 | 0 | 1 | 1 | 1 | 1 | 1 | 1 | 1 | 0 | 1 | 1 | 1 | 1 | 1 | 1 | 1 | 0 | 14 |
| SCF | 1 | 1 | 1 | 1 | 0 | 1 | 1 | 1 | 1 | 1 | 1 | 1 | 1 | 1 | 1 | 1 | 1 | 1 | 1 | 1 | 1 | 18.1 |
| MDN | 1 | 1 | 0 | 0 | 0 | 1 | 1 | 1 | 0 | 0 | 0 | 0 | 0 | 1 | 1 | 1 | 0 | 0 | 0 | 0 | 0 | 6.5 |
| MCN | 1 | 1 | 0 | 0 | 0 | 1 | 1 | 1 | 1 | 1 | 0 | 0 | 0 | 0 | 0 | 1 | 1 | 1 | 0 | 0 | 0 | 9 |
| MDB | 0 | 0 | 0 | 0 | 0 | 1 | 1 | 1 | 1 | 1 | 1 | 1 | 1 | 0 | 0 | 0 | 0 | 0 | 0 | 0 | 0 | 8 |
| MCB | 1 | 1 | 1 | 1 | 1 | 1 | 1 | 1 | 1 | 1 | 1 | 1 | 1 | 1 | 1 | 1 | 1 | 1 | 1 | 1 | 1 | 21 |

The columns in Table 6 were created for two purposes: 1) to serve as grading structure and 2) to give additional information about the regimes at which the valves migrated and their leaflets finctioned properly.

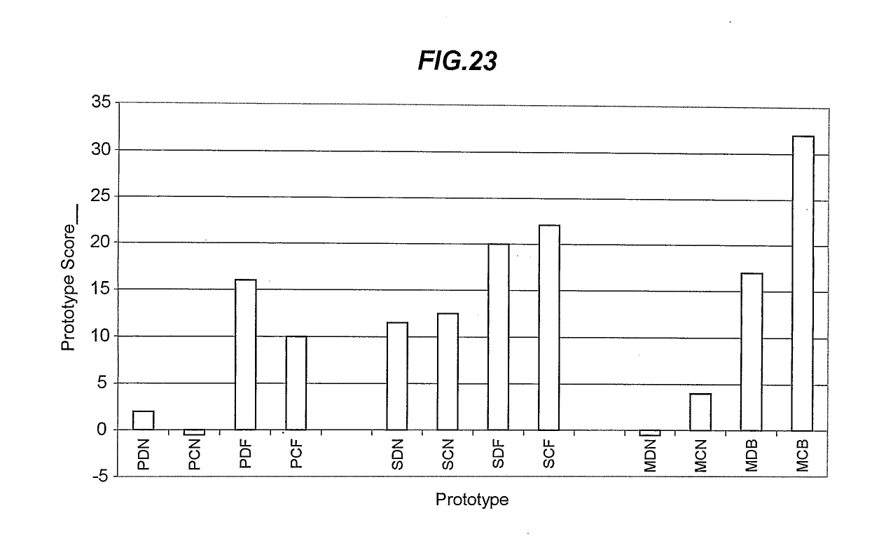

After the computation of the sub-totals for hemodynamic tests all the sub-totals for static tests were added to this column to obtain one final set of numerical values that graded the characteristics of all prototypes. The grand totals for each prototype are shown in FIG. 23 that depicts a comparison of the final grades given to the CBHV prototypes.

The completion of the qualitative studies revealed that the prototype MCB had the highest probability of success among all CBHV prototypes. Other prototypes like the SCF, SDF and MDB had also high scores in the decision matrix, but occasional problems with deployment and leaflet operation led to lower totals than the MCB prototype.

The MCB, in addition to being rated with high attachment levels and consistent leaflet operation, it was considered to require a simpler deployment strategy than all the Pioneer and Simplified models. Although simplicity of deployment was not considered a crucial screening factor at this stage of the project, the future creation of a delivery system will demand the simplest mechanisms of attachment and deployment.

One of the most important results from the qualitative tests was that valves with double coaptation leaflets had considerably higher failure probability than valves with leaflets of central coaptation; that was the main reason why the MDB prototype could not obtain higher grades despite being designed with the same stent structure as the MCB.

Results of the Quantitative Session: The Mcb Performance

Using readings for pressure and flow combined, several parameters were calculated to evaluate the performance of the valves. The following is a list of such parameters.
Forward mean pressure drop takes into account the mean value of the pressure gradient after the valve is opened and positive flow is passing through it.
Mean valvular flow resistance is a parameter calculated from the flow rate and the mean pressure drop; it quantifies the ability of the valve to oppose blood flow.
Backflow per stroke is considered as the portion of fluid that returns to the ventricular chamber during the closing of the valve. Also known as closing volume.
Flow leakage per stroke is a measure of the volume that goes into the ventricle when the valve is closed. It is closely related to the backflow.
Stroke volume is the amount of fluid that passed through the valve during each cardiac cycle; it was used to calculate the percentage of backflow and leakage of the valves.

The information obtained from each test was tabulated as shown in Tables 6-10; these tables represent a summary of the hemodynamic results since they only contain readings obtained at the most representative flow rates—4, 6, 8, 10 and 12 L/min. Some of the testing conditions shown in the following five tables do not show numerical values from the experiment; such situation was produced because some of the cardiovascular regimes required flow and heart rates exceeded the measuring range of the pressure transducers.

The cardiovascular regimes used during the test included extreme conditions at 150 and 180 bpm. Although in some of these extreme conditions measurements for pressure and flow were recorded, they were not included in the comparative analysis of valve performance among the valves. These extreme conditions were mainly used to evaluate the ability of the MCB prototypes to remain attached to the aortic root.

TABLE 7
Hemodynamic results for the natural porcine aortic valve at the most representative cardiovascular regimes.

| NATURAL VALVE | | TOTALS | TOTALS | TOTALS | TOTALS | TOTALS | TOTALS |
|---|---|---|---|---|---|---|---|
| ACKNOWLEDGE FILES | | 50 BPM | 70 BPM | 90 BPM | 120 BPM | 150 BPM | 180 BPM |
| NOMINAL FLOW IN (L/min) | | 4 | 6 | 8 | 10 | 10 | 12 |
| MEAN FLOW RATE | L/min | 3.51 | 5.55 | 7.41 | 9.76 | 9.20 | 11.32 |
| TRANSDUCER OFFSET | L/min | 0.42 | 0.42 | 0.42 | 0.42 | 0.42 | 0.42 |
| STROKE VOLUME | ml | 76.30 | 83.37 | 85.33 | 82.77 | 62.40 | 62.70 |
| VENTRICULAR CONDITIONS | | | | | | | |
| TRANSDUCER OFFSET | mmHg | 2.43 | 2.43 | 2.43 | 2.43 | 2.43 | 2.43 |
| MAXIMUM PRESSURE | mmHg | 205.30 | 193.50 | 178.40 | 229.40 | 167.80 | 267.63 |
| MINIMUM PRESSURE | mmHg | −8.81 | −16.58 | −17.80 | −30.10 | −20.00 | −24.30 |
| AORTIC CONDITIONS | | | | | | | |
| TRANSDUCER OFFSET | mmHg | 13.23 | 13.23 | 13.23 | 13.23 | 13.23 | 13.23 |
| MAXIMUM PRESSURE | mmHg | 193.90 | 193.20 | 178.20 | 216.10 | 150.70 | 189.80 |
| MINIMUM PRESSURE | mmHg | 34.63 | 25.90 | 16.90 | 52.22 | 34.30 | 66.10 |
| VALVE PERFORMANCE | | | | | | | |
| FORWARD MEAN PRESSURE | mmHg | 13.55 | 29.86 | 34.09 | 73.04 | 68.74 | 111.72 |
| DROP | | | | | | | |
| MEAN FLOW RESISTANCE | mmHg · min/L | 3.86 | 5.38 | 4.60 | 7.48 | 7.47 | 9.87 |
| FLOW LEAKAGE PER STROKE | ml | 2.40 | 1.47 | 0.77 | 0.20 | 0.40 | 0.17 |
| % FLOW LEAKAGE PER | | 3.14% | 1.76% | 0.90% | 0.24% | 0.64% | 0.27% |
| STROKE | | | | | | | |
| BACKFLOW PER STROKE | ml | 2.87 | 2.47 | 2.27 | 1.30 | 1.13 | 0.57 |
| % BACKFLOW PER STROKE | | 3.76% | 2.96% | 2.65% | 1.57% | 1.82% | 0.90% |

TABLE 8
Hemodynamic results for the first of the MCB prototypes at the most representative cardiovascular regimes.

| MCB 1 | | TOTALS | TOTALS | TOTALS | TOTALS | TOTALS | TOTALS |
|---|---|---|---|---|---|---|---|
| ACKNOWLEDGE FILES | | 50 BPM | 70 BPM | 90 BPM | 120 BPM | 150 BPM | 180 BPM |
| NOMINAL FLOW IN (L/min) | | 4 | 6 | 8 | 10 | 10 | 12 |
| MEAN FLOW RATE | L/min | 4.43 | 6.42 | 8.40 | 10.24 | 10.22 | 11.78 |
| TRANSDUCER OFFSET | L/min | −0.45 | −0.45 | −0.45 | −0.45 | −0.45 | −0.45 |
| STROKE VOLUME | ml | 99.20 | 97.30 | 97.07 | 90.13 | 70.47 | 86.97 |
| VENTRICULAR CONDITIONS | | | | | | | |
| TRANSDUCER OFFSET | mmHg | 7.35 | 7.35 | 7.35 | 7.35 | 7.35 | 7.35 |
| MAXIMUM PRESSURE | mmHg | 213.80 | 237.70 | 239.30 | 271.30 | 271.30 | 271.30 |
| MINIMUM PRESSURE | mmHg | −16.10 | −23.18 | −25.40 | −32.70 | −27.10 | −29.90 |
| AORTIC CONDITIONS | | | | | | | |
| TRANSDUCER OFFSET | mmHg | 9.52 | 9.52 | 9.52 | 9.52 | 9.52 | 9.52 |
| MAXIMUM PRESSURE | mmHg | 197.90 | 206.70 | 205.60 | 219.60 | 178.24 | 227.03 |
| MINIMUM PRESSURE | mmHg | 9.09 | 15.10 | 16.76 | 40.80 | 43.02 | 63.50 |
| VALVE PERFORMANCE | | | | | | | |
| FORWARD MEAN | mmHg | 38.21 | 57.97 | 66.60 | 116.13 | 122.76 | 115.71 |
| PRESSURE DROP | | | | | | | |
| MEAN FLOW RESISTANCE | mmHg · min/L | 8.63 | 9.03 | 7.93 | 11.34 | 12.01 | 9.82 |
| FLOW LEAKAGE PER | ml | 5.10 | 2.60 | 1.73 | 3.10 | 0.70 | 4.97 |
| STROKE | | | | | | | |
| % FLOW LEAKAGE PER | | 5.14% | 2.67% | 1.79% | 3.44% | 1.00% | 5.71% |
| STROKE | | | | | | | |
| BACKFLOW PER STROKE | ml | 4.53 | 3.37 | 2.90 | 2.03 | 1.83 | 1.67 |
| % BACKFLOW PER STROKE | | 4.57% | 3.46% | 2.99% | 2.26% | 2.60% | 1.92% |

TABLE 9
Hemodynamic results for the second of the MCB prototypes at the most representative cardiovascular regimes.

| MCB 2 | | TOTALS | TOTALS | TOTALS | TOTALS | TOTALS | TOTALS |
|---|---|---|---|---|---|---|---|
| ACKNOWLEDGE FILES | | 50 BPM | 70 BPM | 90 BPM | 120 BPM | 150 BPM | 180 BPM |
| NOMINAL FLOW IN (L/min) | | 4 | 6 | 8 | 10 | 10 | 12 |
| MEAN FLOW RATE | L/min | 4.66 | 6.36 | 8.62 | 10.90 | 10.16 | 10.98 |
| TRANSDUCER OFFSET | L/min | −0.45 | −0.45 | −0.45 | −0.45 | −0.45 | -0.45 |
| STROKE VOLUME | ml | 100.30 | 96.10 | 102.23 | 98.57 | 70.57 | 66.73 |
| VENTRICULAR CONDITIONS | | | | | | | |
| TRANSDUCER OFFSET | mmHg | 7.35 | 7.35 | 7.35 | 7.35 | 7.35 | 7.35 |
| MAXIMUM PRESSURE | mmHg | 192.20 | 224.50 | 253.80 | 271.30 | 252.50 | 271.20 |
| MINIMUM PRESSURE | mmHg | −15.60 | −22.80 | −24.50 | −31.40 | −24.60 | -28.81 |
| AORTIC CONDITIONS | | | | | | | |
| TRANSDUCER OFFSET | mmHg | 9.52 | 9.52 | 9.52 | 9.52 | 9.52 | 9.52 |
| MAXIMUM PRESSURE | mmHg | 196.60 | 215.90 | 273.30 | 226.25 | 166.32 | 175.40 |
| MINIMUM PRESSURE | mmHg | 0.00 | 20.70 | 27.90 | 30.70 | 33.50 | 49.70 |
| VALVE PERFORMANCE | | | | | | | |
| FORWARD MEAN | mmHg | 32.94 | 65.12 | 77.48 | 115.49 | 111.50 | 133.14 |
| PRESSURE DROP | | | | | | | |
| MEAN FLOW RESISTANCE | mmHg · min/L | 7.07 | 10.24 | 8.99 | 10.60 | 10.97 | 12.13 |
| FLOW LEAKAGE PER | ml | 4.00 | 1.67 | 5.40 | 3.20 | 1.07 | 3.50 |
| STROKE | | | | | | | |
| % FLOW LEAKAGE PER | | 3.99% | 1.74% | 5.28% | 3.25% | 1.51% | 5.25% |
| STROKE | | | | | | | |
| BACKFLOW PER STROKE | ml | 4.30 | 3.97 | 1.53 | 2.67 | 1.53 | 1.97 |
| % BACKFLOW PER STROKE | | 4.29% | 4.13% | 1.50% | 2.70% | 2.17% | 2.95% |

TABLE 10
Hemodynamic results for the third of the MCB prototypes at the most representative
cardiovascular regimes. Pressure readings for 180 bpm reached the transducer limit.

| MCB 3 | | TOTALS | TOTALS | TOTALS | TOTALS | TOTALS | TOTALS |
|---|---|---|---|---|---|---|---|
| ACKNOWLEDGE FILES | | 50 BPM | 70 BPM | 90 BPM | 120 BPM | 150 BPM | 180 BPM |
| NOMINAL FLOW IN (L/min) | | 4 | 6 | 8 | 10 | 10 | 12 |
| MEAN FLOW RATE | L/min | 4.05 | 6.01 | 7.67 | 10.33 | 10.30 | 0.00 |
| TRANSDUCER OFFSET | L/min | −0.45 | −0.45 | −0.45 | −0.45 | −0.45 | −0.45 |
| STROKE VOLUME | ml | 88.93 | 94.13 | 90.87 | 88.83 | 71.00 | #DIV/0I |
| VENTRICULAR CONDITIONS | | | | | | | |
| TRANSDUCER OFFSET | mmHg | 7.35 | 7.35 | 7.35 | 7.35 | 7.35 | 7.35 |
| MAXIMUM PRESSURE | mmHg | 186.30 | 259.05 | 229.00 | 271.25 | 271.25 | #DIV/0I |
| MINIMUM PRESSURE | mmHg | −13.40 | −22.70 | −23.80 | −30.20 | −26.40 | #DIV/0I |
| AORTIC CONDITIONS | | | | | | | |
| TRANSDUCER OFFSET | mmHg | 9.52 | 9.52 | 9.52 | 9.52 | 9.52 | 9.52 |
| MAXIMUM PRESSURE | mmHg | 176.60 | 227.03 | 199.80 | 208.90 | 172.04 | #DIV/0I |
| MINIMUM PRESSURE | mmHg | 13.18 | 38.70 | 24.45 | 35.60 | 40.20 | #DIV/0I |
| VALVE PERFORMANCE | | | | | | | |
| FORWARD MEAN | mmHg | 32.86 | 68.64 | 65.49 | 114.37 | 119.67 | #DIV/0I |
| PRESSURE DROP | | | | | | | |
| MEAN FLOW RESISTANCE | mmHg · min/L | 8.11 | 11.42 | 8.54 | 11.07 | 11.62 | #DIV/0I |
| FLOW LEAKAGE PER | ml | 3.07 | 3.37 | 2.43 | 0.90 | 0.90 | #DIV/0I |
| STROKE | | | | | | | |
| % FLOW LEAKAGE PER | | 3.45% | 3.58% | 2.68% | 1.01% | 1.27% | #DIV/0I |
| STROKE | | | | | | | |
| BACKFLOW PER STROKE | ml | 4.87 | 4.00 | 2.90 | 2.00 | 1.87 | #DIV/0I |
| % BACKFLOW PER STROKE | | 5.47% | 4.25% | 3.19% | 2.25% | 2.63% | #DIV/0I |

TABLE 11
Hemodynamic results for the control valve at the most representative cardiovascular regimes.

| CONTROL VALVE SUTURED TO VESSEL | | TOTALS | TOTALS | TOTALS | TOTALS | TOTALS | TOTALS |
|---|---|---|---|---|---|---|---|
| ACKNOWLEDGE FILES | | 50 BPM | 70 BPM | 90 BPM | 120 BPM | 150 BPM | 180 BPM |
| NOMINAL FLOW IN (L/min) | | 4 | 6 | 8 | 10 | 10 | 12 |
| MEAN FLOW RATE | L/min | 4.20 | 6.13 | 8.11 | 10.40 | 11.12 | 11.12 |
| TRANSDUCER OFFSET | L/min | −0.11 | −0.11 | −0.11 | −0.11 | −0.11 | −0.11 |
| STROKE VOLUME | ml | 87.83 | 92.03 | 94.53 | 89.53 | #DIV/0I | #DIV/0I |
| VENTRICULAR CONDITIONS | | | | | | | |
| TRANSDUCER OFFSET | mmHg | 8.24 | 8.24 | 8.24 | 8.24 | 8.24 | 8.24 |
| MAXIMUM PRESSURE | mmHg | 189.70 | 255.40 | 259.50 | 270.30 | #DIV/0I | #DIV/0I |
| MINIMUM PRESSURE | mmHg | −15.80 | −25.60 | −26.60 | −31.30 | #DIV/0I | #DIV/0I |
| AORTIC CONDITIONS | | | | | | | |
| TRANSDUCER OFFSET | mmHg | 10.50 | 10.50 | 10.50 | 10.50 | 10.50 | 10.50 |
| MAXIMUM PRESSURE | mmHg | 185.80 | 222.90 | 196.90 | 189.90 | #DIV/0I | #DIV/0I |
| MINIMUM PRESSURE | mmHg | 18.10 | 38.20 | 26.50 | 46.80 | #DIV/0I | #DIV/0I |
| VALVE PERFORMANCE | | | | | | | |
| FORWARD MEAN | mmHg | 39.16 | 73.65 | 86.83 | 131.46 | #DIV/0I | #DIV/0I |
| PRESSURE DROP | | | | | | | |
| MEAN FLOW RESISTANCE | mmHg · min/L | 9.65 | 18.14 | 21.39 | 32.38 | 0.00 | 0.00 |
| FLOW LEAKAGE PER | ml | 3.33 | 1.80 | 0.90 | 0.63 | #DIV/0I | #DIV/0I |
| STROKE | | | | | | | |
| % FLOW LEAKAGE PER | | 3.80% | 1.96% | 0.95% | 0.71% | #DIV/0I | #DIV/0I |
| STROKE | | | | | | | |
| BACKFLOW PER STROKE | ml | 2.70 | 3.43 | 3.23 | 0.70 | #DIV/0I | #DIV/0I |
| % BACKFLOW PER STROKE | | 3.07% | 3.73% | 3.42% | 0.78% | #DIV/0I | #DIV/0I |

Numerical values for pressure difference, closing volume and flow leakage summarized in the previous tables were plotted to facilitate comparison in the performance of the devices. As previously mentioned, regimes above 120 bpm were not included in the analysis of the performance of the devices and the natural valve.

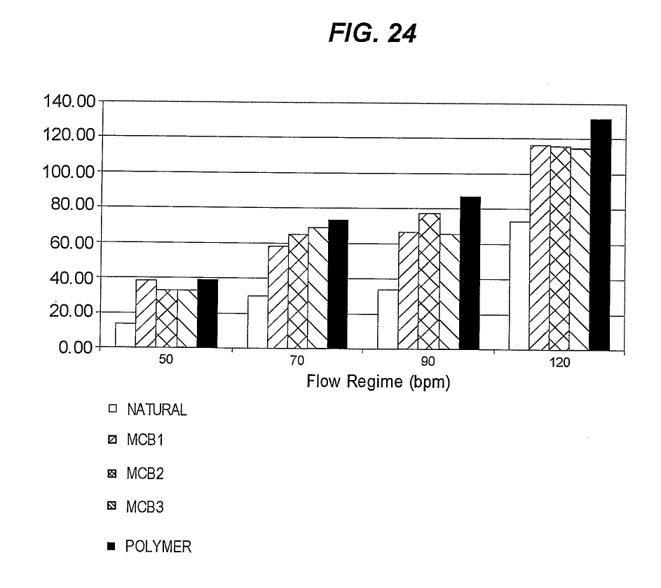

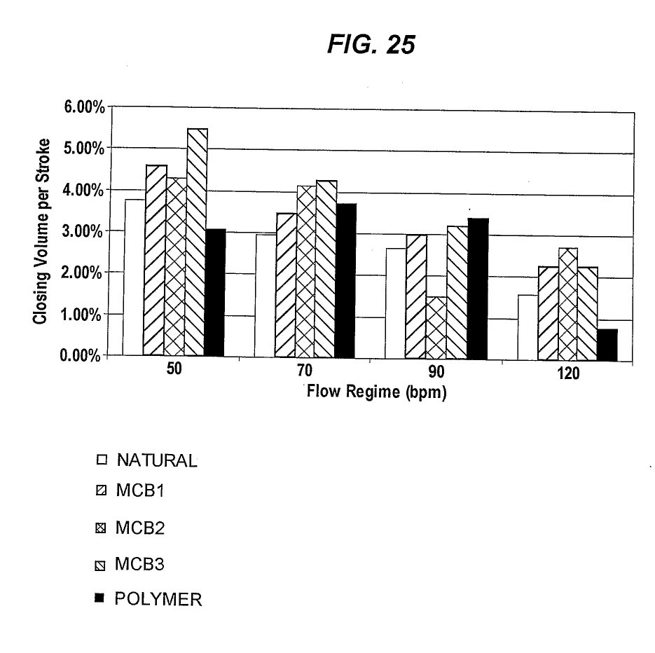

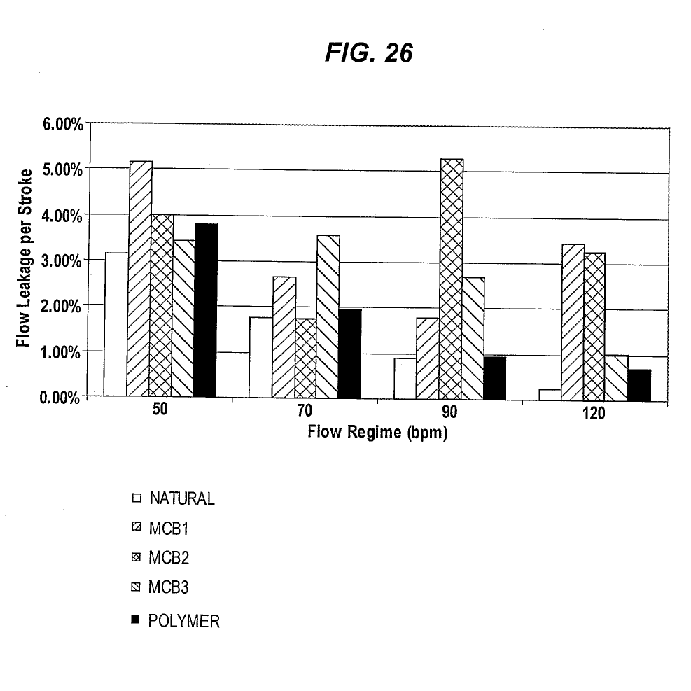

FIGS. 24-26 show the summarized results for hemodynamic performance of the tested valves. Three sample devices of the MCB prototypes were tested along with the natural porcine aortic valve and a traditional polymer valve.

Conclusions to Quantitative Tests

The complete set of hemodynamic experiments done during the quantitative session was a successful experiment; not only because of its numerical outcomes that allowed the comparison of the valves' performance, but also because it was the first time a hemodynamic test for prosthetic heart valves included the interaction of a natural aortic root.

The traditional setup for hemodynamic tests was designed in such a way that it required all prosthetic valves to have rigid stents so they could be assembled to the system. With the creation of collapsible structures like the CBHV'S, the traditional testing setup was no longer useful. Some important design requirements like vessel attachment or valve migration could not be tested without a piece of natural tissue integrated to the system.

The modification of the system to allow the testing of collapsible heart valves offered a very practical and reliable alternative for the testing of the CBHV'S; but not without revealing some system trade-offs. The most important trade-off that was observed after the modification of the traditional system was that the compliance levels of the system were changed with the modified setup; such changes altered the pressure waveforms by enlarging their ranges of oscillation.

Another trade-off of the modified setup was that the incorporation of the porcine aortic root limited the testing capabilities of the system to valve diameters of up to 19 mm. The reduction in diameter of the valves in conjunction with the vessel fixture was found to increase the overall pressure readings in the system; such pressure readings were more likely to reach the limits of the pressure transducers under higher flow regimes.

Limitations on the pressure measurements at higher flow regimes were the main reason for some of the incomplete test results. This situation in addition to observations on the performance of the Valves at lower flow regimes led to the decision of restricting the hemodynamic analysis to the moderate flow regimes only.

The analysis of the four moderate flow regimes for all valves was concentrated on the values for pressure difference, percentage of closing volume and percentage of flow leakage. The pressure difference in the natural porcine aortic valve was lower than the pressure difference values of the prosthetic devices.

The values for pressure difference among the prosthetic valves revealed that the MCB prototypes were less obstructive to the flow than the polymer valve; the deformable structure of the MCB prototypes allowed the devices to follow the expansion of the aortic root during systole; such change in diameter of the vessel and the valve facilitated the transit of the fluid across the valve. In the case of the polymer valve, its structure was rigid and any changes to the vessel diameter during systole were impeded by the sutured attachment created around the valve.

Measurements in closing volume showed a rather different scenario from the one observed in the analysis of the pressure difference: the performance of the natural valve was not consistently better than the performance of the prosthetic devices; this observation was particularly true in the case of the polymer valve; because, the default configuration of its leaflets was the closed position. Valves that are manufactured with their leaflets in their closed position require less backflow to shut off the valve; that is why in the case of the polymer valve it was observed that the percentage of volume required to close the valve had values that were more competitive than the values for its pressure difference. The improved performance in closing volume of the polymer valve in some cases (for 50 and 120 bpm) was even better than the one observed in the natural valve.

An analysis of variance and post-hoc tests of the closing volume confirmed previous observations. Tests showed that the closing volumes measured at 120 bpm and 90 bpm were not significantly different from each other and that the polymer valve had a significantly different closing volume than the rest of the valves.

Results in Flow leakage showed the highest variability among all tests; such variability was observed specially within the MCB valves along the tested flow regimes. Results in flow leakage of the natural valve and the control valve (the polymer valve) were relatively consistent along different flow regimes; this observation led to the conclusion that changes in the flow regime can interfere with the ability of the valve to prevent leakage.

However, statistical analysis of the leakage of the valves showed that the differences between the natural valve, the control valve and the MCB valves were not enough evidence to conclude that the natural valve and the control valve were significantly better than the MCB prototypes.

Although certain heart valve constructions have been described herein in accordance with the teachings of the present disclosure, the scope of patent coverage is not limited thereto. On the contrary, this patent covers all embodiments of the teachings of the disclosure that fairly fall within the scope of permissible equivalents.

1. A human heart valve replacement comprising:
a collapsible stent formed from at least one length of wire, the wire having a series of turns forming a spring-like stent wall; and
at least one leaflet attached to the stent;
wherein the stent wall is collapsible in a radial direction such that a contracted diameter of the heart valve is smaller than an expanded diameter of the heart valve,
wherein the stent wall is spring biased to the expanded diameter, and
wherein the heart valve is sufficiently collapsible to be disposed within a catheter for insertion into a human heart.

2. The human heart valve replacement of claim 1, including three leaflets attached to the stent.

3. The human heart valve replacement of claim 2, wherein the leaflets are arranged in a double coaptation configuration.

4. The human heart valve replacement of claim 2, wherein the leaflets are arranged in a central coaptation configuration.

5. The human heart valve replacement of claim 1, wherein the at least one leaflet comprises a fabric selected from the group consisting of DACRON®, Polyester and Polypropylene.

6. The human heart valve replacement of claim 1, wherein the at least one leaflet is less than approximately 280 microns thick.

7. The human heart valve replacement of claim 1, wherein the at least one leaflet comprises a material having a square thread pattern.

8. The human heart valve replacement of claim 7, wherein the material is arranged such that the leaflet has a higher elasticity along lines of coaptation and lower elasticity along a blood flow direction.

9. The human heart valve replacement of claim 1, further including a forward migration retainer extending from the stent wall.

10. The human heart valve replacement of claim 9, wherein the forward migration retainer comprises a hook.

11. The human heart valve replacement of claim 1, further including a backflow migration retainer extending from the stent wall.

12. The human heart valve replacement of claim 11, wherein the forward migration retainer comprises a hook.

13. The human heart valve replacement of claim 12, further including a forward migration retainer that comprises a hook.

14. The human heart valve replacement of claim 11, wherein the backflow migration retainer is adapted to engage a natural leaflet in a heart such that the natural leaflet is disposed between the stent wall and the backflow migration retainer and the backflow migration retainer is disposed between the natural leaflet and a vessel wall.

15. The human heart valve replacement of claim 1, wherein the wire is a nitinol wire.

16. The human heart valve replacement of claim 1, wherein the ratio between the expanded diameter and the contracted diameter is approximately 3:1.

17. The human heart valve replacement of claim 1, wherein the stent wall is cylindrical in shape.

18. The human heart valve replacement of claim 1, wherein ends of the wire are joined with a hypodermic sleeve.

19. The human heart valve replacement of claim 1, wherein the expanded diameter is in the range of approximately 18 mm to approximately 27 mm.

20. The human heart valve replacement of claim 1, wherein the contracted diameter is in the range of approximately 6 mm to approximately 9 mm.

21. The human heart valve replacement of claim 1, wherein a length of the stent is in the range of approximately 12 mm to approximately 24 mm.

22. The human heart valve replacement of claim 1, wherein the stent and the at least one leaflet are coated with a biocompatible material.

23. The human heart valve replacement of claim 22, wherein the biocompatible material is SIBS.

24. The human heart valve replacement of claim 23, wherein the at least one leaflet is coated with a 20 ml solution of SIBS for 12 hours and dried at 80 degrees C.

25. A human heart valve replacement comprising:
a collapsible stent formed from at least one length of nitinol wire, the nitinol wire having a series of turns forming a spring-like stent wall;
a forward migration retainer extending from one end of the stent wall, the forward migration retainer being formed from a loop of the nitinol wire and adapted to engage a vessel wall to prevent migration of the replacement human heart valve in a blood flow direction;
a backflow migration retainer extending from another end of the stent wall, the backflow migration retainer being formed from a loop of the nitinol wire and adapted to engage a natural leaflet to prevent migration of the replacement human heart valve in a direction opposite to blood flow;
three leaflets having a thickness of less than 280 microns attached to the stent, the three leaflets forming a central coaptation arrangement;
wherein the stent wall is collapsible in a radial direction such that a contracted diameter of the replacement human heart valve is smaller than an expanded diameter of the replacement human heart valve,
wherein the stent wall is spring biased to the expanded diameter,
wherein the replacement human heart valve is sufficiently collapsible to be disposed within a catheter for insertion into a human heart, and
wherein the heart valve is coated with a boicompatable material.

26. A method of forming a stent for a replacement human heart valve, the method comprising:
attaching an end of a wire to a stent plate and attaching the other end of the wire to a tensor;
stretching the wire along a path determined by a plurality of pins on the stent plate;
thermally treating the stretched wire;
attaching the wire to a second plate;
bending portions of the wire to form a forward migration retainer and a backflow migration retainer;
thermally treating the stretched wire;
bending the stent into a substantially cylindrical shape; and
fixing the ends of the wire together within a hypodermic tube.

27. A method of forming a leaflet for a replacement human heart valve, the method comprising;
providing a sheet of material having a thickness of less than 280 microns;
soaking the sheet of material in a 20 ml solution of SIBS;
drying the sheet of material for approximately 12 hours at approximately 80 degrees C.; and
folding the sheet of material into one of a double coaptation arrangement and a central coaptation arrangement.

28. A method of inserting a human heart valve replacement into a human heart, the method comprising:
providing a replacement human heart valve comprising:
a collapsible stent formed from a length of wire, the wire having a series of turns forming a spring-like stent wall; and
a leaflet attached to the stent;
wherein the stent wall is collapsible in a radial direction such that a contracted diameter of the replacement human heart valve is smaller than an expanded diameter of the replacement human heart valve, the stent wall is spring biased to the expanded diameter, and the replacement human heart valve is sufficiently collapsible to be disposed within a catheter for insertion into a human heart;
compressing the stent to a diameter less than that of a catheter;
inserting the replacement human heart valve into the catheter;
inserting the catheter into the human heart; and
expanding the replacement human heart valve into an operational position in the human heart.

29. A leaflet for an artificial human heart valve comprising:
a sheet of woven fabric material;
wherein the sheet of woven material is both peripherally and centrally coaptable.

30. The leaflet of claim 29 wherein the woven fabric material is selected from the group consisting of DACRON®, Polyester and Polypropylene.

31. The leaflet of claim 29 wherein the sheet of woven fabric material has two plies that form a double coaptation configuration.

32. The leaflet of claim 29, wherein a first ply coapts peripherally and a second ply coapts centrally.

33. The leaflet of claim 29, wherein the woven fabric material is folded and the free ends of the woven fabric material are downstream of the folded end of the woven fabric material in a direction of blood flow.

34. The leaflet of claim 29, wherein the sheet of woven material has a single centrally coapatable ply.

35. The leaflet of claim 34, wherein the ply coapts both centrally and peripherally.

36. The leaflet of claim 35, wherein the ply coapts centrally at one end and peripherally at another end.

37. The leaflet of claim 29, wherein the woven fabric material has a square weave pattern.

38. The leaflet of claim 29, wherein the woven fabric material is coated with a biocompatible material.

39. The leaflet of claim 38, wherein the biocompatible material is SIBS.

40. The leaflet of claim 29, wherein the woven fabric material is less than approximately 280 microns thick.

41. The leaflet of claim 29, wherein the woven fabric material has a higher elasticity along lines of coaptation and a lower elasticity along a flow direction.

42. The leaflet of claim 29, wherein three like pieces of woven fabric material form a substantially cylindrical shape, yet the peripheral coaptation provides a flexible in the peripheral geometry.
